(19) United States
(12) Patent Application Publication
(10) Pub. No.: US 2016/0348190 A1
(43) Pub. Date: Dec. 1, 2016

(54) SYSTEMS AND METHODS FOR CONTINUOUS FLOW PCR SYSTEMS

(71) Applicant: STOKES BIO LIMITED, Limerick (IE)

(72) Inventors: Mauro Aguanno, Singapore (SG); David Kinahan, Kildare (IE); Mark Korenke, Richmond, VA (US); Ryan Talbot, Stamford, CT (US); Mark Gaughran, Co. Clare (IE); Matthew Lough, Co. Westmeath (IE)

(21) Appl. No.: 15/136,679

(22) Filed: Apr. 22, 2016

Related U.S. Application Data
(63) Continuation of application No. 14/110,667, filed on Dec. 18, 2013, now abandoned.
(60) Provisional application No. 61/473,263, filed on Apr. 8, 2011.

(51) Int. Cl. C12Q 3/00; B01L 7/00; G01N 35/08; C12Q 1/68

(52) U.S. Cl. CPC C12Q 3/00; C12Q 1/686; B01L 7/5255; G01N 35/08; B01L 2300/0627; G01N 2035/00366

(57) ABSTRACT

A liquid handling system of a PCR system is instructed to obtain a matrix of samples and reagents for a PCR experiment. A fluid pumping system of the PCR system is instructed to maintain a continuous flow of a transport fluid through a plurality of micro-channels that allows the mixture of the samples and the reagents producing a plurality of mixed sample droplets. One or more post-bridge detection values are received from a post-bridge detection system of the PCR system for each mixed sample droplet to determine if the mixed sample droplet is mixed correctly. A thermocycler of the PCR system is instructed to maintain one or more temperatures for cycling the temperature of the plurality of mixed sample droplets. One or more endpoint detection values are received from an endpoint detection system of the PCR system for each mixed sample droplet to analyze the PCR experiment.

### CROSS-REFERENCE TO RELATED APPLICATIONS

This application is a continuation of U.S. application Ser. No. 14/110,667, filed Dec. 18, 2013, which is a 371 of International Application No. PCT/US2012/032582, filed Apr. 6, 2012, which claims the benefit of Provisional Application No. 61/473,263, filed Apr. 8, 2011, all of which are incorporated herein by reference in their entirety.

### INTRODUCTION

Polymerase chain reaction (PCR) systems or thermocyclers typically include a sample block, a heated cover, and heating and cooling elements. These components are then controlled or monitored by an onboard control system. Real-time PCR systems or thermocyclers generally also include an optical detection system for detecting electromagnetic radiation emitted by one or more probes attached to a nucleic acid sample. Real-time PCR systems can additionally include an external computer or control system for controlling and monitoring system components and analyzing data produced by the optical detection system.

Current standard PCR systems and real-time PCR systems are well-based systems. These systems receive samples in a sample support device that includes a plurality of wells. The samples are prepared or mixed with reagents before being loaded into the PCR system. The PCR system then cycles the temperatures of the samples in the wells. Additionally, real-time PCR systems monitor the samples in the wells for electromagnetic or fluorescent emissions.

As the uses and need for genetic and genomic information have increased, so has the need for PCR amplification and analysis. In particular, it has become increasingly important to improve the throughput of PCR systems. Although each generation of PCR systems can cycle the temperatures of samples slightly faster, the technology has not kept up with the performance improvements of other genetic and genomic analysis instruments. For example, deoxyribonucleic acid (DNA) sequencing instruments are advancing to the point where sample preparation and PCR amplification are the most limiting steps in terms of time and cost for sequencing experiments.

In addition, the reliance of current PCR systems on well-based technology limits the overall throughput of these systems. Current systems can cycle the temperatures of samples in approximately 40 minutes. Using the largest well-based sample support device with 384 wells, therefore, produces a maximum overall sample throughput of about 500 samples per hour. Further, current PCR systems receive samples already prepared or mixed in the sample support device. Therefore these systems are dependent on the time consuming and sometimes manual step of well-based sample preparation.

### SUMMARY

A system, method, and computer program product are provided for high throughput polymerase chain reaction (PCR) amplification and analysis. The system includes a PCR system and a processor in communication with the PCR system. The method includes steps that use a PCR system and a processor.

The computer program product includes a non-transitory and tangible computer-readable storage medium. The computer-readable storage medium includes a program with instructions that are executed on a processor. The instructions executed on the processor perform a method for high throughput PCR amplification and analysis. The method includes providing a system of distinct software modules that includes a liquid handling module, a fluid pumping module, a post-bridge detection module, a thermocycler module, and an endpoint detection module.

In the system and method, a processor sends instructions to and receives data values from a number of components of the PCR system. The processor instructs a liquid handling system to obtain a plurality of samples and a plurality of reagents for a PCR experiment. The processor instructs a fluid pumping system to maintain a continuous flow of a transport fluid through a plurality of micro-channels. The continuous flow allows the fluid pumping system to receive the plurality of samples and the plurality of reagents from the liquid handling system as droplets in the plurality of micro-channels. The continuous flow also allows the fluid pumping system to mix the plurality of samples and the plurality of reagents using the geometry of the plurality of micro-channels producing a plurality of mixed sample droplets in the plurality of micro-channels.

The processor receives from a post-bridge detection system of the PCR system one or more post-bridge detection values for each mixed sample droplet of the plurality of mixed sample droplets to determine if each mixed sample droplet is mixed correctly. The processor instructs a thermocycler of the PCR system to maintain one or more temperatures for cycling the temperature of the plurality of mixed sample droplets in the plurality of micro-channels. Finally, the processor receives from an endpoint detection system of the PCR system one or more endpoint detection values for each mixed sample droplet of the plurality of mixed sample droplets to analyze the PCR experiment.

In various embodiments, the processor instructs the liquid handling system to pipette samples from a first sample support device located on a first tray of the liquid handling system, pipette assay reagents from a second sample support device located on a second tray of the liquid handling system, and pipette a master mix reagent from a vessel.

In various embodiments, the one or more post-bridge detection values include a time stamp of the mixed sample droplet. In various embodiments, the one or more post-bridge detection values include the intensity of electromagnetic radiation absorbed or reflected by the mixed sample droplet. In various embodiments, the one or more post-bridge detection values include a first intensity of electromagnetic radiation emitted by a first dye of a sample of the mixed sample droplet, a second intensity of electromagnetic radiation emitted by a second dye of an assay reagent of the mixed sample droplet, and a third intensity of electromagnetic radiation emitted by a third dye of a master mix reagent of the mixed sample droplet.

In various embodiments, the processor further instructs the liquid handling system to re-sample a sample and an assay reagent of the mixed sample droplet, if the processor determines from the one or more post-bridge detection values that the mixed sample droplet is mixed incorrectly.

In various embodiments, the one or more endpoint detection values include a location of a micro-channel and a spectral intensity detected from the micro-channel.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is a schematic diagram showing a system for high throughput polymerase chain reaction (PCR) amplification and analysis, in accordance with various embodiments.

FIG. 3 is an exemplary flowchart showing a method for high throughput PCR amplification and analysis, in accordance with various embodiments.

FIG. 4 is a schematic diagram of a system that includes one or more distinct software modules that perform a method for high throughput PCR amplification and analysis, in accordance with various embodiments.

FIG. 5 is a schematic diagram of the software architecture for a continuous flow PCR system, in accordance with various embodiments.

FIG. 6 is a flowchart showing a system initialization method, in accordance with various embodiments.

FIG. 7 is a flowchart showing a method for issuing a transmission control protocol/internet protocol (TCP/IP) command, in accordance with various embodiments.

FIG. 8 is a flowchart showing a first portion of a method for issuing a run command, in accordance with various embodiments.

FIG. 9 is a flowchart showing a second portion of a method for issuing a run command, in accordance with various embodiments.

FIG. 10 is a flowchart showing a third portion of a method for issuing a run command, in accordance with various embodiments.

FIG. 11 is a flowchart showing a system shutdown method, in accordance with various embodiments.

FIG. 12 is a flowchart showing a method for handling errors, in accordance with various embodiments.

FIG. 13 is a schematic diagram of a flap valve opening method, in accordance with various embodiments.

FIG. 14 is a schematic diagram of a liquid/plate handling system, in accordance with various embodiments.

FIGS. 15A-F is a flowchart showing a first portion of a method for plate stacking, in accordance with various embodiments.

FIGS. 16A-B is a flowchart showing a second portion of a method for plate stacking, in accordance with various embodiments.

FIGS. 17A-B is a flowchart showing a third portion of a method for plate stacking, in accordance with various embodiments.

FIG. 18 is a flowchart showing a method for liquid handling initialization, in accordance with various embodiments.

FIGS. 19A-B is a flowchart showing a method for liquid handling, in accordance with various embodiments.

FIG. 20 is a flowchart showing a method for liquid handling shutdown, in accordance with various embodiments.

FIG. 21 is a state diagram showing the relationships among post-bridge methods, in accordance with various embodiments.

FIG. 22 is a flowchart showing a first portion of a post-bridge initialization method, in accordance with various embodiments.

FIG. 23 is a flowchart showing a second portion of a post-bridge initialization method, in accordance with various embodiments.

FIG. 24 is a flowchart showing a post-bridge pre run method, in accordance with various embodiments.

FIG. 25 is a flowchart showing a first portion of a post-bridge run method, in accordance with various embodiments.

FIG. 26 is a flowchart showing a second portion of a post-bridge run method, in accordance with various embodiments.

FIG. 27 is a flowchart showing a third portion of a post-bridge run method, in accordance with various embodiments.

FIG. 28 is a flowchart showing a post-bridge run end method, in accordance with various embodiments.

FIG. 29 is a flowchart showing a post-bridge shutdown method, in accordance with various embodiments.

FIG. 30 is a schematic diagram showing tray and position waypoints, in accordance with various embodiments.

FIG. 31 is a schematic diagram showing how files are transferred between a graphical user interface (GUI) and an instrument, in accordance with various embodiments.

FIG. 32 is a flowchart showing a method for uploading a file using a file transfer protocol (FTP) server, in accordance with various embodiments.

FIG. 33 is a schematic diagram of a side view of a system for detecting spectral and spatial information in a continuous flow PCR system, in accordance with various embodiments.

FIG. 34 is a schematic diagram of a top view of a system for detecting spectral and spatial information in a continuous flow PCR system, in accordance with various embodiments.

FIG. 35 is a schematic diagram of a three-dimensional view of a tube array plate, in accordance with various embodiments.

FIG. 36 is a schematic diagram of a top view of a tube array plate, in accordance with various embodiments.

FIG. 37 is a schematic diagram of a side view of a tube array plate, in accordance with various embodiments.

FIG. 38 is a flowchart showing a method for detecting spectral and spatial information in a continuous PCR system, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

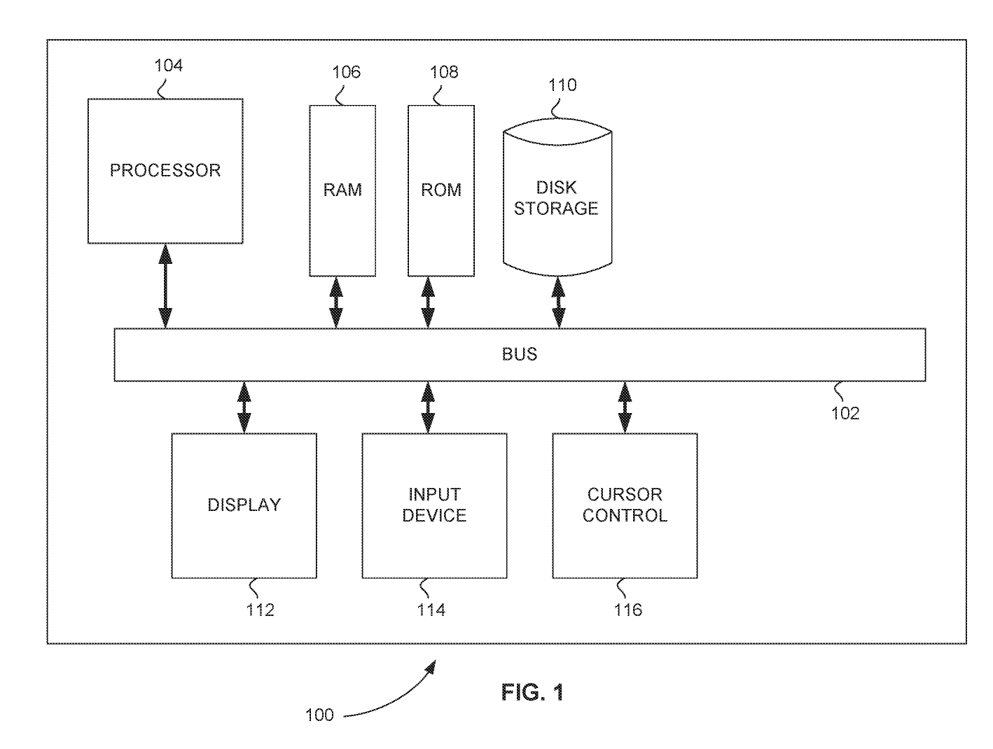

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random access memory (RAM) or other dynamic storage device, coupled to bus 102 for determining base calls, and instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively hard-wired circuitry may be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium may take many forms, including but not limited to, non-volatile media, volatile media, and transmission media. Non-volatile media includes, for example, optical or magnetic disks, such as storage device 110. Volatile media includes dynamic memory, such as memory 106. Transmission media includes coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a non-transitory and tangible computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Systems and Methods of Data Processing

### Continuous Flow PCR System

As described above, the reliance of current polymerase chain reaction (PCR) systems on well-based technology can limit the overall throughput of these systems. Also, current PCR systems receive samples already prepared or mixed in the sample support device. Therefore these systems are dependent on the time consuming and sometimes manual step of well-based sample preparation.

In various embodiments, systems and methods for continuous flow PCR amplification and analysis are used. These systems and methods significantly increase the sample throughput of a PCR experiment and reduce the limitations imposed by well-based technology. In particular, systems and methods for continuous flow PCR essentially eliminate a sample preparation step by incorporating it into the PCR process.

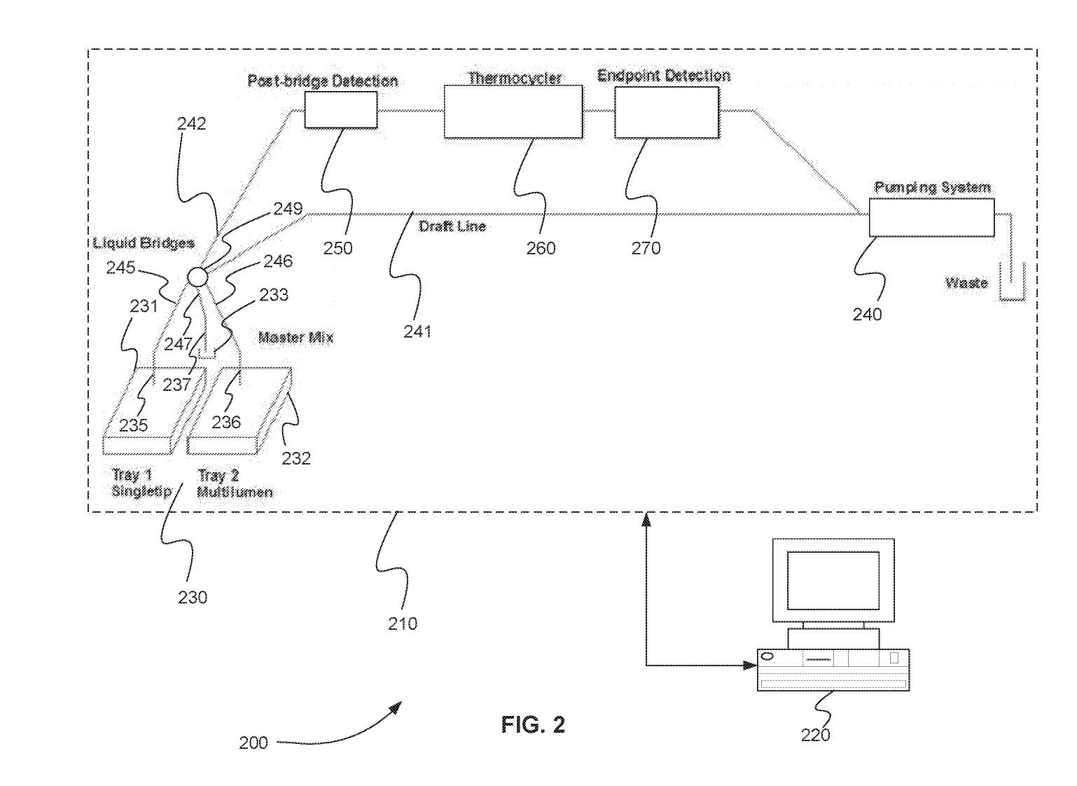

FIG. 2 is a schematic diagram showing a system 200 for high throughput PCR amplification and analysis, in accordance with various embodiments. System 200 includes PCR system 210 and processor 220. PCR system 210, in turn, includes liquid handling system 230, fluid pumping system 240, post-bridge detection system 250, thermocycler 260, and endpoint detection system 270.

Processor 220 is in communication with PCR system 210. Processor 220 can include, but is not limited to, a computer, a microprocessor, a microcontroller, an application specific integrated circuit (ASIC), or any device capable of executing instructions and sending and receiving data or control communications.

Processor 220 instructs liquid handling system 230 to obtain a plurality of samples and a plurality of reagents for a PCR experiment. In various embodiments, processor 220 instructs liquid handling system 230 to pipette samples from a first sample support device (not shown) located on tray 231 of liquid handling system 230, pipette assay reagents from a second sample support device (not shown) located on tray 232 of liquid handling system 230, and pipette a master mix reagent from vessel 233.

In various embodiments, a sample support device may be a glass or plastic slide with a plurality of sample regions. Some examples of a sample support device may include, but are not limited to, a multi-well plate, such as a standard microtiter 96-well, a 384-well plate, or a microcard, or a substantially planar support, such as a glass or plastic slide. The sample regions in various embodiments of a sample support device may include depressions, indentations, ridges, and combinations thereof, patterned in regular or irregular arrays formed on the surface of the substrate.

Processor 220 instructs fluid pumping system 240 to maintain a continuous flow of a transport fluid through a plurality of micro-channels. The transport fluid or oil is a passive buffer for carrying samples around system 200. FIG. 2 shows a single micro-channel of the plurality of micro-channels. This single micro-channel or tube includes draft line 241 and thermocycler line 242. Draft line 241 is used to bleed off excess transport fluid and maintain the continuous flow of a transport fluid through the micro-channel at a constant flow rate. Thermocycler line 242 is used to carry mixed samples through system 200.

Processor 220 instructs fluid pumping system 240 to maintain a continuous flow of a transport fluid in order to receive the plurality of samples and the plurality of reagents from liquid handling system 230 as droplets in the plurality of micro-channels. The continuous flow of a transport fluid by fluid pumping system 240 draws a sample droplet from tip 235 of liquid handling system 230 up through line 245 of fluid pumping system 240. Similarly, the continuous flow of a transport fluid by fluid pumping system 240 draws an assay reagent droplet from tip 236 of liquid handling system 230 up through line 246 of fluid pumping system 240 and draws a master mix reagent droplet from tip 237 of liquid handling system 230 up through line 247 of fluid pumping system 240, for example.

Further, the continuous flow of a transport fluid by fluid pumping system 240 causes the plurality of samples and the plurality of reagents to be mixed using the geometry of the plurality of micro-channels. This results in a plurality of mixed sample droplets in the plurality of micro-channels. The geometry of the plurality of micro-channels that causes the plurality of samples and the plurality of reagents to be mixed is a junction or liquid bridge of micro-channels, for example.

Junction 249 is an exemplary liquid bridge for mixing samples and reagents for a single micro-channel. Lines 245, 246, and 247 meet at junction 249. Through precise timing control, processor 220 instructs liquid handling system 230 to select sample, assay reagent, and master mix droplets using tips 235, 236, and 247 at specific times so that fluid pumping system 240 draws these droplets to junction 249 at the same time. Because sample, assay reagent, and master mix droplets reach junction 249 simultaneously, they are mixed as they are moving with the continuous flow of transport fluid. The mixture produces a mixed sample droplet. This mixed sample droplet leaves junction 249 and enters thermocycler line 242. The mixed sample droplet continues moving with the continuous flow of transport fluid at a constant flow rate in thermocycler line 242.

In order to determine if each mixed sample droplet is mixed correctly, processor 220 receives one or more post-bridge detection values for each mixed sample droplet of the plurality of mixed sample droplets from post-bridge detection system 250. Post-bridge detection system 250, for example, detects mixed sample droplets in thermocycler line 242 at precise time steps selected by processor 220. In various embodiments, post-bridge detection system 250 is an optical system that includes one or more sources of illumination and one or more cameras. In various embodiments, one camera is used and the one or more post-bridge detection values include the intensity of electromagnetic radiation absorbed or reflected by each mixed sample droplet.

In various embodiments, three cameras are used by post-bridge detection system 250. The one or more post-bridge detection values received by processor 220 then include a first intensity of electromagnetic radiation emitted by a first dye of a sample of each mixed sample droplet, a second intensity of electromagnetic radiation emitted by a second dye of an assay reagent of each mixed sample droplet, and a third intensity of electromagnetic radiation emitted by a third dye of a master mix reagent of the mixed sample droplet. In various embodiments, the one or more post-bridge detection values also include a time stamp of the mixed sample droplet so the processor can identify the sample and reagents used to create the mixed sample droplet.

In various embodiments, processor 220 instructs liquid handling system 230 to re-sample a sample and an assay reagent of a mixed sample droplet, if processor 220 determines from the one or more post-bridge detection values that the mixed sample droplet is mixed incorrectly. In other words, if processor 220 determines that the one or more post-bridge detection values that the mixed sample droplet are not indicative of a proper mixture, processor instructs liquid handling system 230 to re-sample the sample and reagents used to create the mixed sample droplet.

After a mixed sample droplet of the plurality of mixed sample droplets is analyzed by post-bridge detection system 250, it moves to thermocycler 260. Processor 220 instructs thermocycler 260 to maintain one or more temperatures for cycling the temperature of the plurality of mixed sample droplets in the plurality of micro-channels. In various embodiments, thermocycler 260 includes two or more heating and cooling elements that are instructed to maintain two or more temperatures. As each mixed sample droplet is moved among the two or more heating and cooling elements, the temperature of the mixed sample droplet is cycled.

Finally, processor 220 receives from endpoint detection system 270 one or more endpoint detection values for each mixed sample droplet of the plurality of mixed sample droplets. Processor 220 uses the one or more endpoint detection values to analyze the PCR experiment. In various embodiments, endpoint detection system 270 is also an optical detection system. Endpoint detection system 270 is a hyperspectral imaging system that determines both spatial and spectral information, for example. Therefore, in various embodiments, the one or more endpoint detection values include the location of a micro-channel and a spectral intensity value detected from that micro-channel. The location of the micro-channel allows processor 220 to identify the mixed sample droplet and the spectral intensity value detected provides a measure of the result of the PCR experiment.

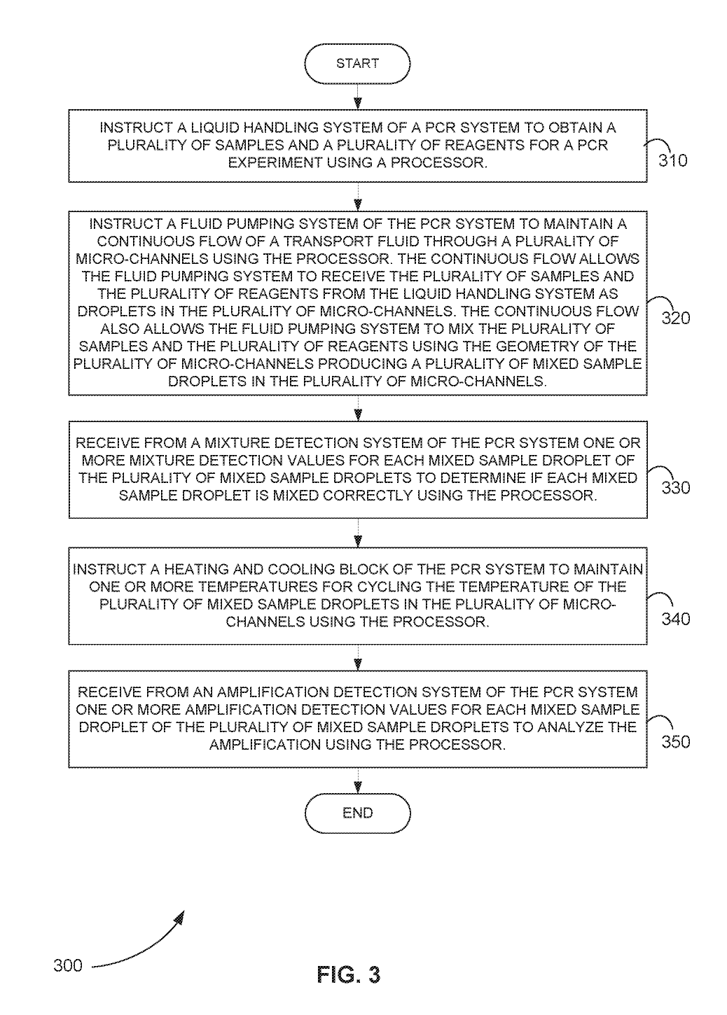

FIG. 3 is an exemplary flowchart showing a method 300 for high throughput PCR amplification and analysis, in accordance with various embodiments.

In step 310 of method 300, a liquid handling system of a PCR system is instructed to obtain a plurality of samples and a plurality of reagents for a PCR experiment using a processor.

In step 320, a fluid pumping system of the PCR system is instructed to maintain a continuous flow of a transport fluid through a plurality of micro-channels using the processor. The continuous flow allows the fluid pumping system to receive the plurality of samples and the plurality of reagents from the liquid handling system as droplets in the plurality of micro-channels. The continuous flow also allows the fluid pumping system to mix the plurality of samples and the plurality of reagents using the geometry of the plurality of micro-channels. Mixing the plurality of samples and the plurality of reagents produces a plurality of mixed sample droplets in the plurality of micro-channels.

In step 330, one or more post-bridge detection values are received from a post-bridge detection system of the PCR system for each mixed sample droplet of the plurality of mixed sample droplets to determine if each mixed sample droplet is mixed correctly using the processor.

In step 340, a thermocycler of the PCR system is instructed to maintain one or more temperatures for cycling the temperature of the plurality of mixed sample droplets in the plurality of micro-channels using the processor.

In step 350, one or more endpoint detection values are received from an endpoint detection system of the PCR system for each mixed sample droplet of the plurality of mixed sample droplets to analyze the PCR experiment using the processor.

In various embodiments, a computer program product includes a non-transitory and tangible computer-readable storage medium whose contents include a program with instructions being executed on a processor so as to perform a method for high throughput PCR amplification and analysis. This method is performed by a system that includes one or more distinct software modules.

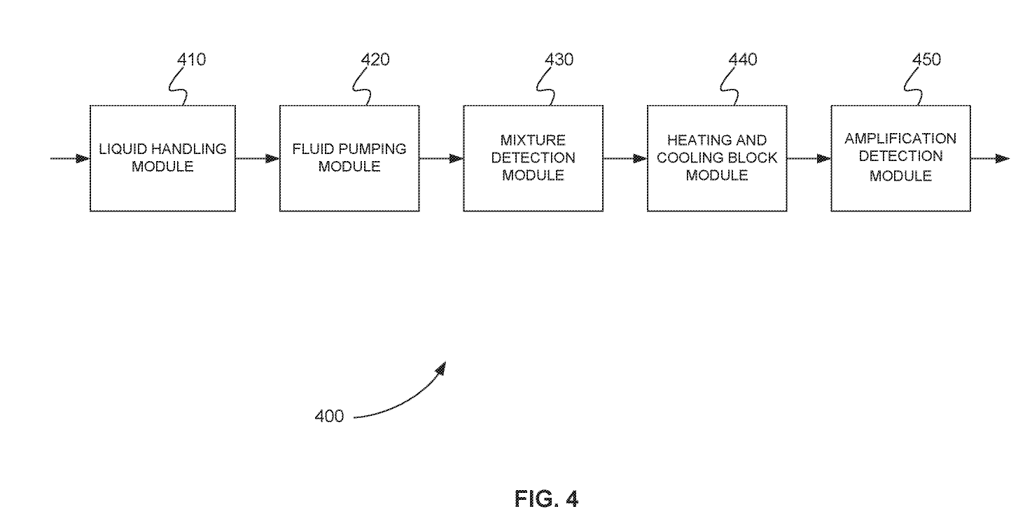

FIG. 4 is a schematic diagram of a system 400 that includes one or more distinct software modules that perform a method for high throughput PCR amplification and analysis, in accordance with various embodiments. System 400 includes liquid handling module 410, fluid pumping module 420, post-bridge detection module 430, thermocycler module 440, and endpoint detection module 450.

Liquid handling module 410 instructs a liquid handling system of a PCR system to obtain a plurality of samples and a plurality of reagents for a PCR experiment.

Fluid pumping module 420 instructs a fluid pumping system of the PCR system to maintain a continuous flow of a transport fluid through a plurality of micro-channels. The continuous flow allows the fluid pumping system to receive the plurality of samples and the plurality of reagents from the liquid handling system as droplets in the plurality of micro-channels. The continuous flow also allows the fluid pumping system to mix the plurality of samples and the plurality of reagents using the geometry of the plurality of micro-channels producing a plurality of mixed sample droplets in the plurality of micro-channels.

Post-bridge detection module 430 receives from a post-bridge detection system of the PCR system one or more post-bridge detection values for each mixed sample droplet of the plurality of mixed sample droplets to determine if each mixed sample droplet is mixed correctly.

Thermocycler module 440 instructs a thermocycler of the PCR system to maintain one or more temperatures for cycling the temperature of the plurality of mixed sample droplets in the plurality of micro-channels.

Endpoint detection module 450 receiving from an endpoint detection system of the PCR system one or more endpoint detection values for each mixed sample droplet of the plurality of mixed sample droplets to analyze the PCR experiment.

### Exemplary Continuous Flow PCR System

An exemplary continuous flow PCR System is a continuous flow 96-line PCR instrument capable of sampling from master-mix, sample and primer/probes simultaneously and mixing these in a micro-channel geometry (Liquid Bridges). The mixed droplets flow downstream to a thermocycler where they are amplified. The droplets then pass a data-acquisition system where their fluorescent intensities are measured.

In order to enable system operation the following software controlled elements are present: fluid pumping system, liquid handling/plate handling system, post-bridge detection, thermocycler, endpoint detection, and ancillary equipment. The fluid pumping system includes five flow sensors, five pumps and more than 40 level sensors and valves. The liquid handling/plate handling system includes a plate stacker, a barcode reader, and a 15 axis sampling unit. The post-bridge detection includes three Basler cameras. The thermocycler includes four 24-line temperature controlled thermocyclers (TCs) each with separate denaturation blocks. The endpoint detection includes one Hamamatsu Orca camera and one laser.

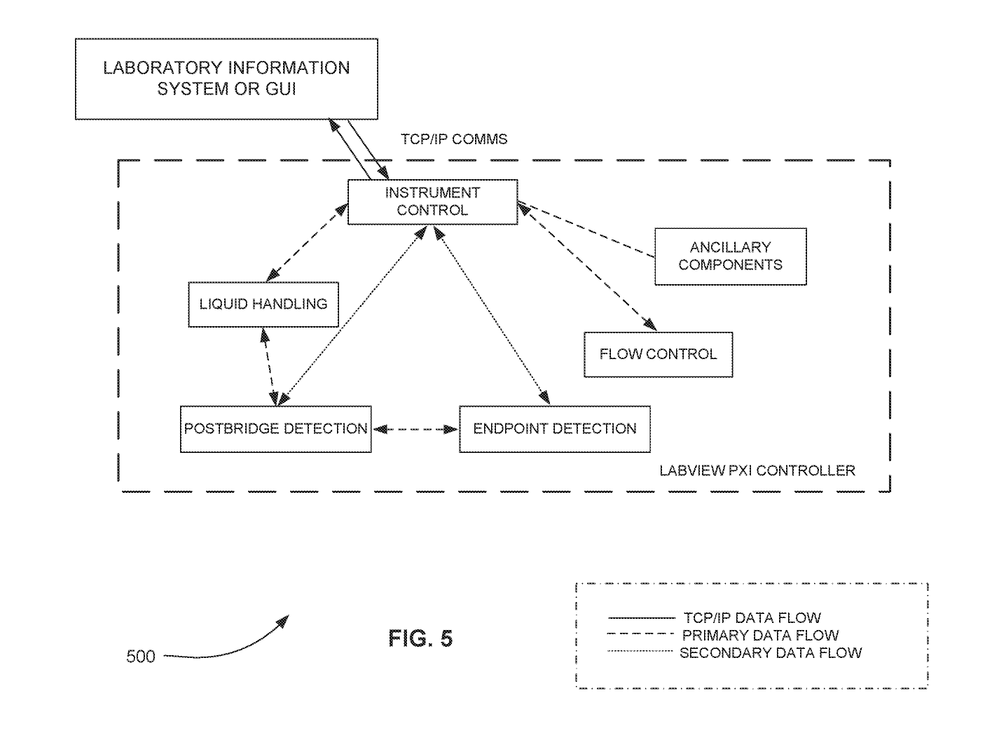

FIG. 5 is a schematic diagram of the software architecture for a continuous flow PCR system, in accordance with various embodiments.

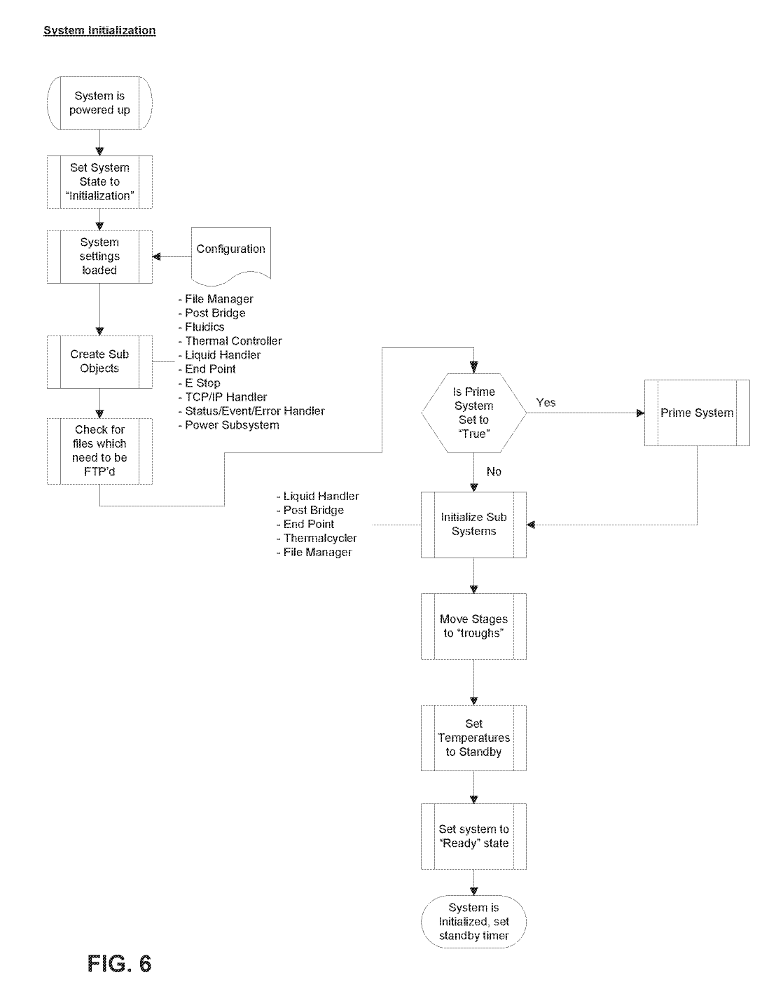

FIG. 6 is a flowchart showing a system initialization method, in accordance with various embodiments.

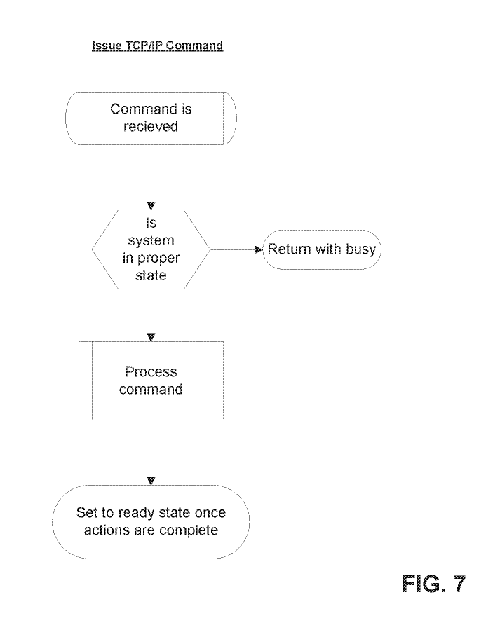

FIG. 7 is a flowchart showing a method for issuing a transmission control protocol/internet protocol (TCP/IP) command, in accordance with various embodiments.

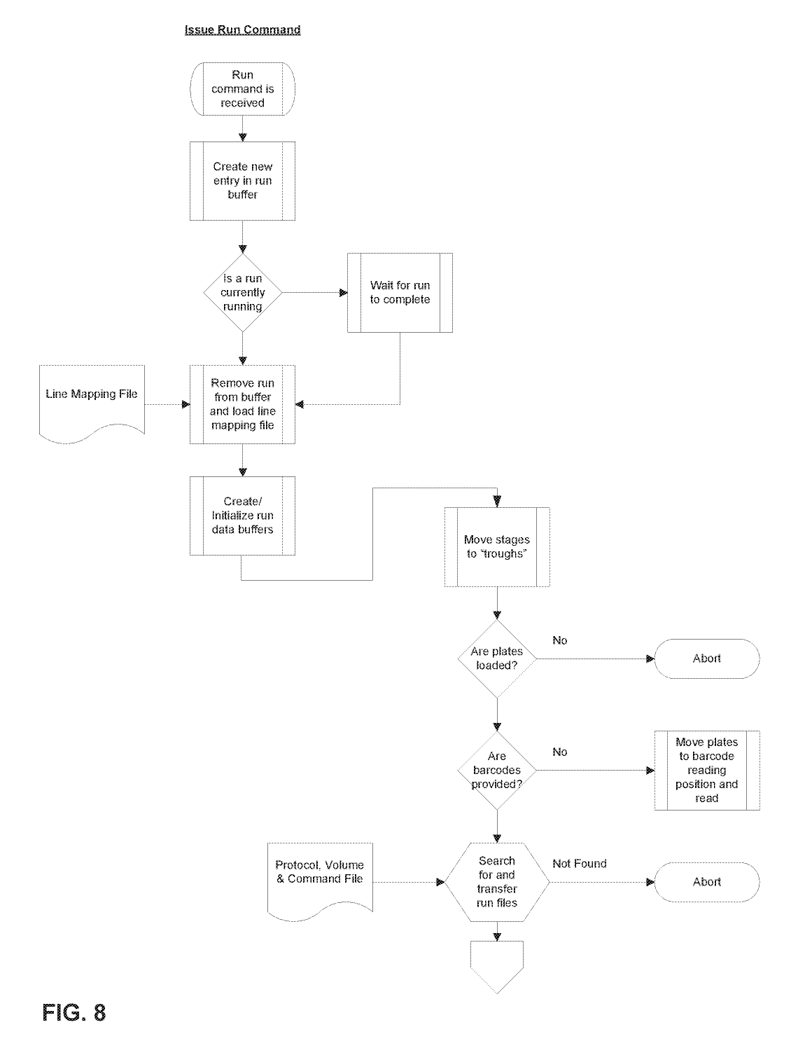

FIG. 8 is a flowchart showing a first portion of a method for issuing a run command, in accordance with various embodiments.

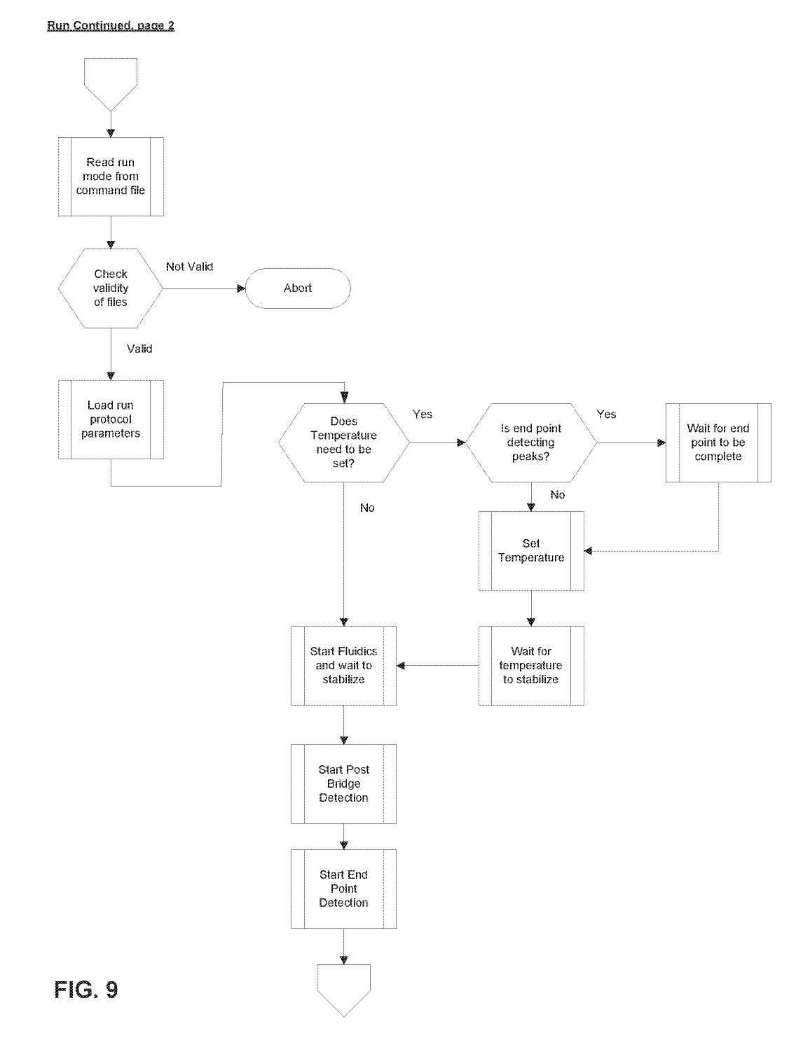

FIG. 9 is a flowchart showing a second portion of a method for issuing a run command, in accordance with various embodiments.

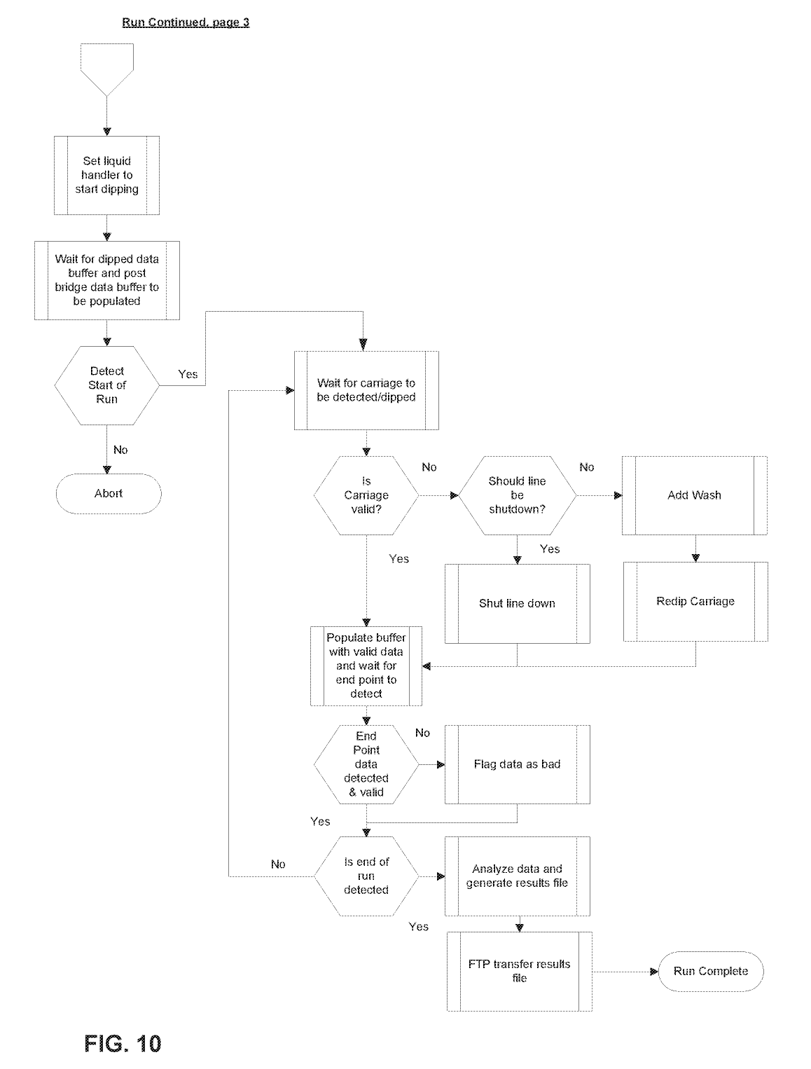

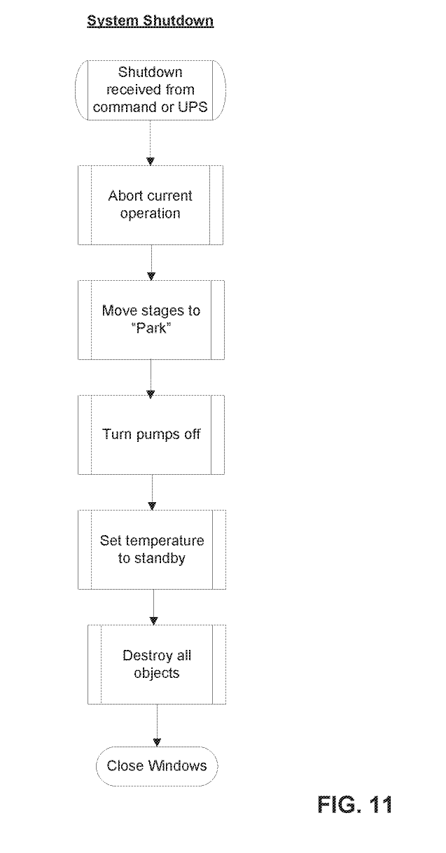

FIG. 10 is a flowchart showing a third portion of a method for issuing a run command, in accordance with various embodiments.

FIG. 11 is a flowchart showing a system shutdown method, in accordance with various embodiments.

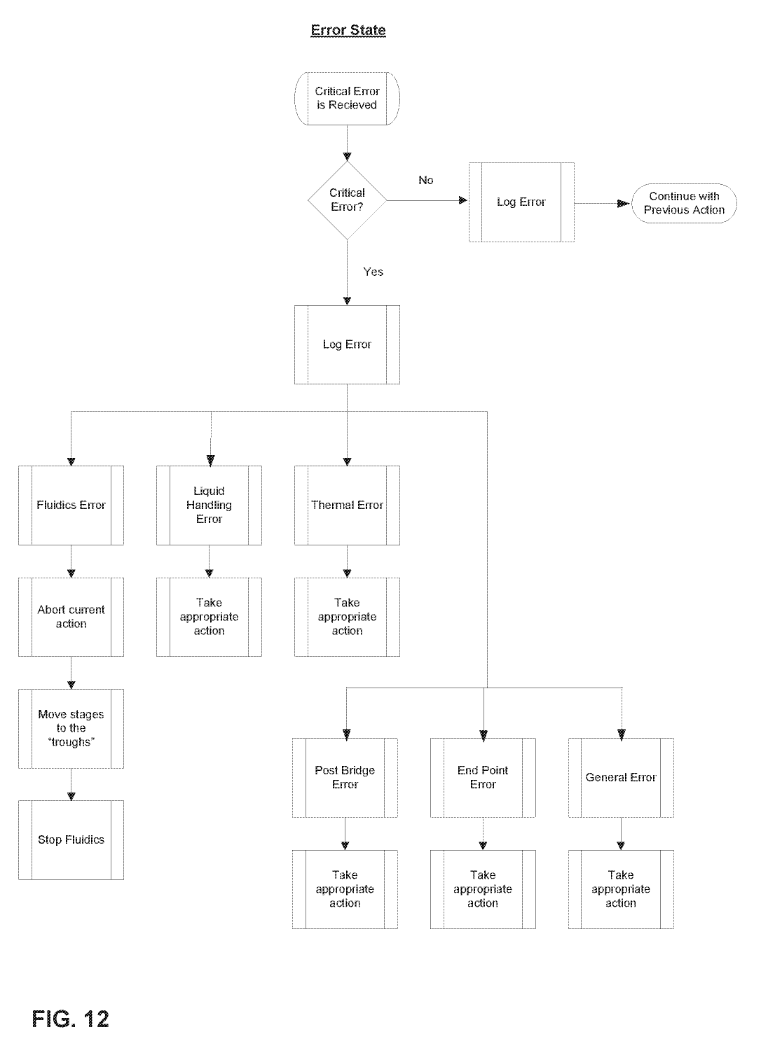

FIG. 12 is a flowchart showing a method for handling errors, in accordance with various embodiments.

### Fluid Pumping System

Referring again to FIG. 2, the system 200 operates under the principal of continuous flow. A constant flow of oil is maintained through the thermocycler (TC line 242) and this flow of oil carries mixed droplets. It is required that the flow upstream of the liquid-bridges (from sample-tips to bridges) be faster than the flow through the thermocycler in order to meet throughput demands. A draft line 241 is fitted to the bridge and bleeds off excess oil. The TC line 242 and the draft line 241 both operate at fixed flow rates. It is required that these lines be controlled as the addition of droplets to the lines increases the pressure drop along each line. The combined flow in the TC line 242 and draft Line 241 equals that of the master-mix, sample and primer-probe lines.

In addition the pumping system incorporates a number of subsystems for priming the system with oil and bleeding it of air. FIG. 2 shows a general schematic (for a single line system) showing the TC Line 242, the Draft Line 241 and where the hardware components are located.

### Sheathing

If a PCR system operates under continuous flow, moving the system through air to move from well-to-well would cause air to be drawn into the system. This is avoided through the use of sheathing/flap valves. These larger bore tubes are fitted around the sampling tubes and wrap them in oil. The continuous flow of oil into the sheathing (driven by 3 independent sheathing pumps) matches (or slightly exceeds) the flow being drawn into the system tips insuring that the continuous flow lines are always wrapped in oil. Hence the tips can move freely from well to well without drawing any air into the system.

### Liquid Handling/Plate Changing

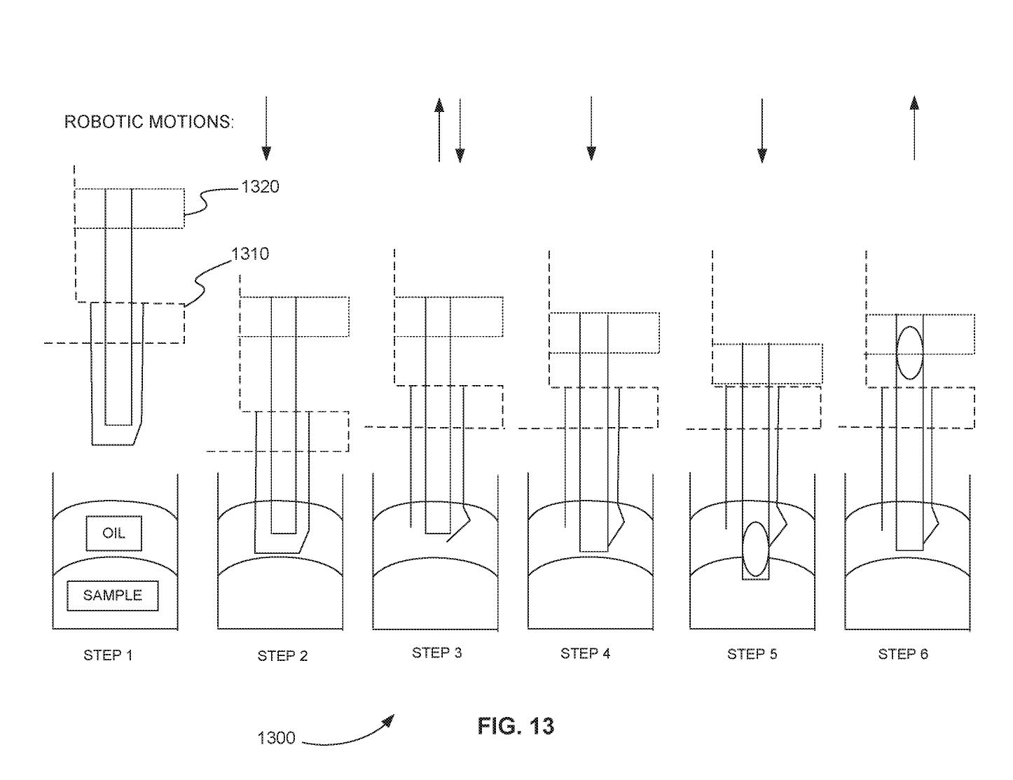

FIG. 13 is a schematic diagram of a flap valve opening method 1300, in accordance with various embodiments. In order to facilitate the use of flap valves/sheathing (which needs to be opened before sampling can take place) the tips are mounted on a double Z-axis. The secondary axis 1320 is mounted on the primary axis 1310. The sheathing/flap valves are mounted on primary axis 1310 while the tips are mounted on secondary axis 1320.

In step 1 of method 1300, in air the robotic head moves over the required wells.

In step 2, primary axis 1310 lowers the tips (sheathing and secondary axis 1320) into the oil overlay which covers the sample in each well.

In step 3, secondary axis 1320 then extends the tips (pushing the valves open) so the tip is over the sample. Simultaneously primary axis 1310 rises by an equal distance. The combined effect is that secondary axis 1320 is stationary in space while primary axis 1310 moves upwards. Combined with the geometry of the flap-valves, this movement allows an extra 30 μl volume of sample be used in each (96-wellplate) well.

In step 4, secondary axis 1320 lowers further into the well and completes opening of the flap valve. The secondary axis 1320 pauses until triggered to sample.

In step 5, at the precise time required, secondary axis 1320 dips into the fluid and draws up approximately 75 nl of fluid (sample/primer-probe, master mix approx. 150 nl). The amount of fluid drawn depends on the flow-rate used and the time the tip is within the fluid.

In step 6, the tip then retracts from the sample and pauses ready to sample again if required. If the next sample is needed from a neighboring well (or a plate-change) the tip retracts into the sheathing and the primary axis 1310 then moves the sampling head out into the air. The sheathing motion is a reverse of the unsheathing motions.

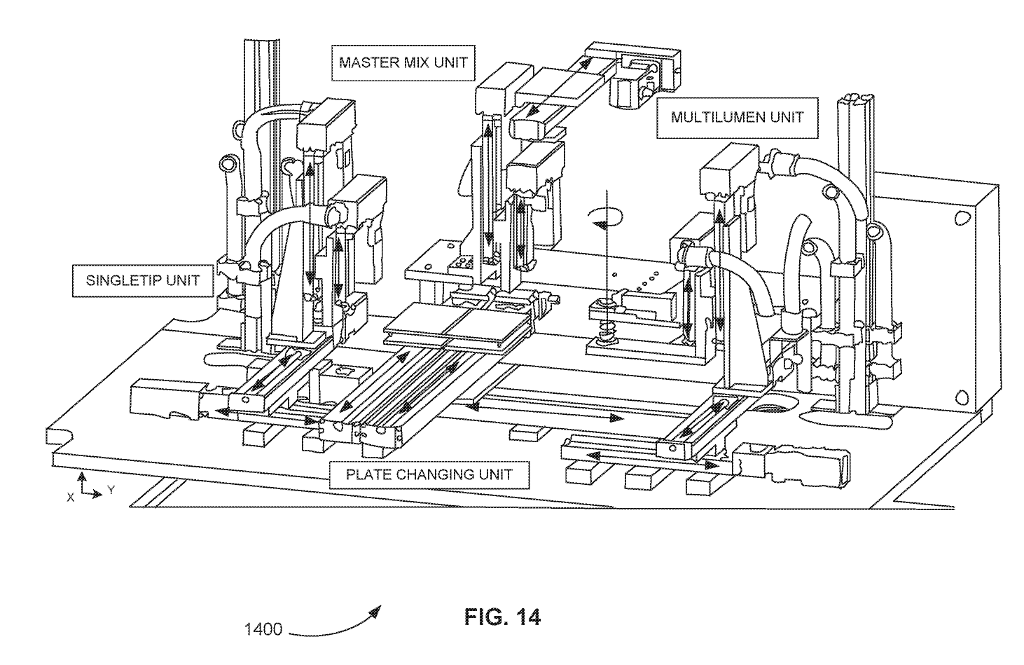

FIG. 14 is a schematic diagram of a liquid/plate handling system 1400, in accordance with various embodiments. In system 1400, the liquid/plate handling provides movement along 15 axes. For reference, system 1400 is divided into three sampling systems and one plate handling system. The directions of motion of each stage are shown by arrows. Note that the sampling arm of the multi-lumen unit is shown. However, for clarity, the sampling arms of the master-mix unit and single-tip unit are rendered invisible. Additionally the master mix unit is mounted on the roof of the enclosure. The individual axes are:
Single-tip Sampling
   X-axis
   Y-axis
   Primary Z-axis (Z1)
   Secondary Z-axis (Z2)
Multi-lumen Sampling
   X-axis
   Y-axis
   Primary Z-axis (Z1)
   Secondary Z-axis (Z2)
   Rotational Axis
Master-mix Sampling
   X-axis
   Primary Z-axis (Z1)
   Secondary Z-axis (Z2)
Plate handling
   Y-axis
   X1-axis (Tray1—Single-tip)
   X2-axis (Tray2—Multi-lumen)

The single-tip system consists of 96 tips each of which can enter a single well on a 96-well or 384-well plate. Therefore system 1400 can sample from a 96-well plate in a single movement or a 384-well plate in four movements. The multi-lumen system consists of four bundles of 24-tips. All 24 lines in each bundle can enter a single well. Each line in the bundle is arrayed against one of the single-tip lines—meeting in a bridge and then flowing into the thermocycler. The Multi-lumen head is mounted on a rotational unit. Therefore through four rotation and dips four wells on Tray 2 (Multi-lumen side) can be arrayed against an entire 96-well plate. Similarly 16 robotic movements (four multi-lumen rotations times four single-tip movements) can permit four wells on Tray 2 be arrayed against an entire 384-well plate.

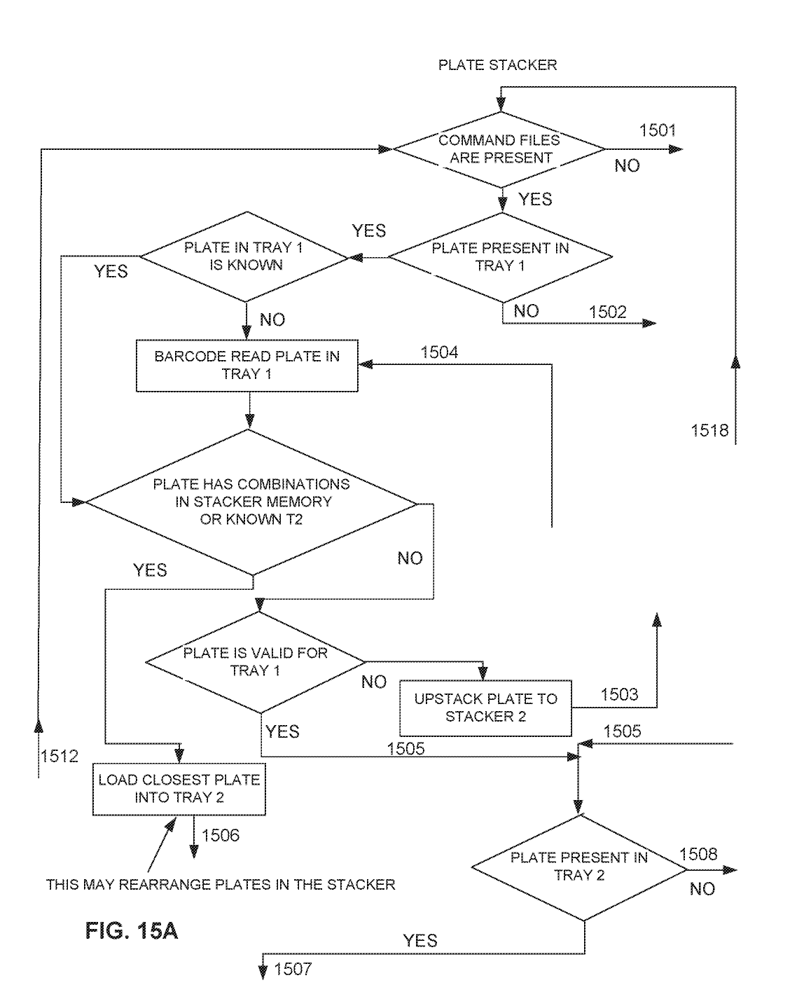

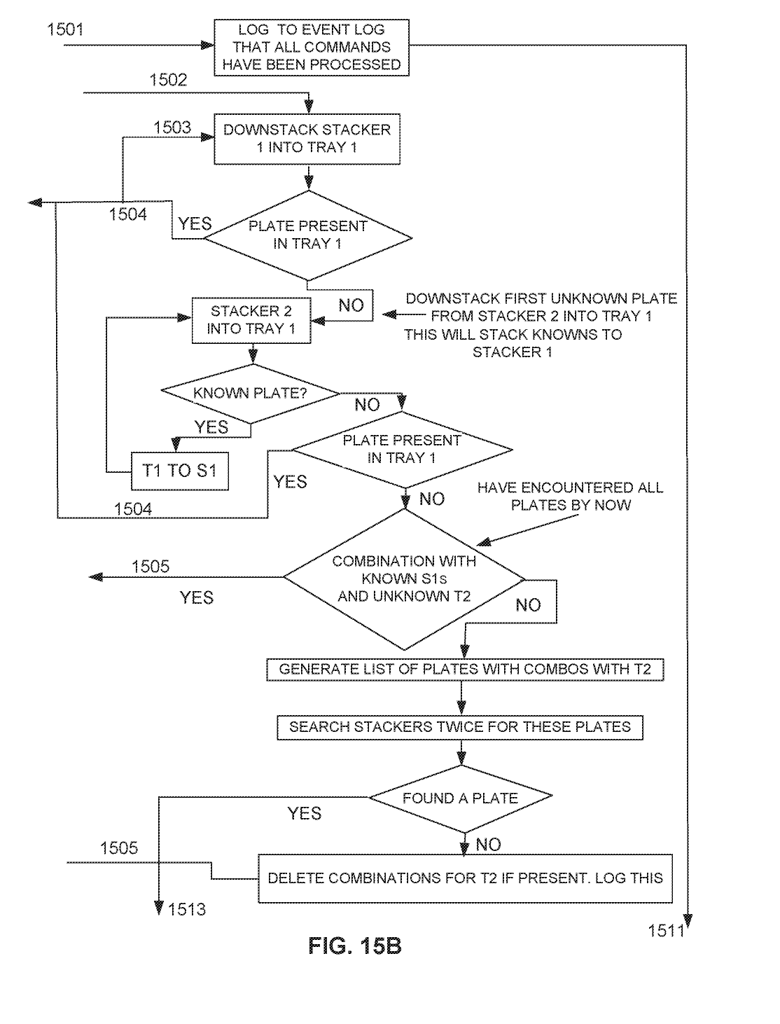

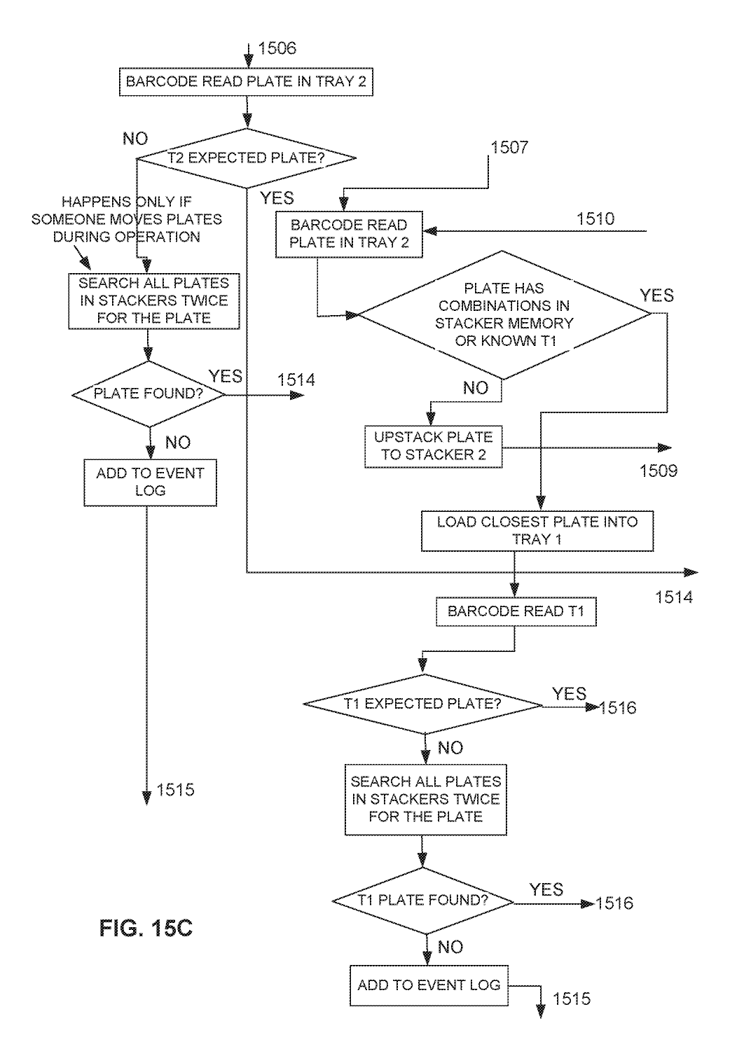

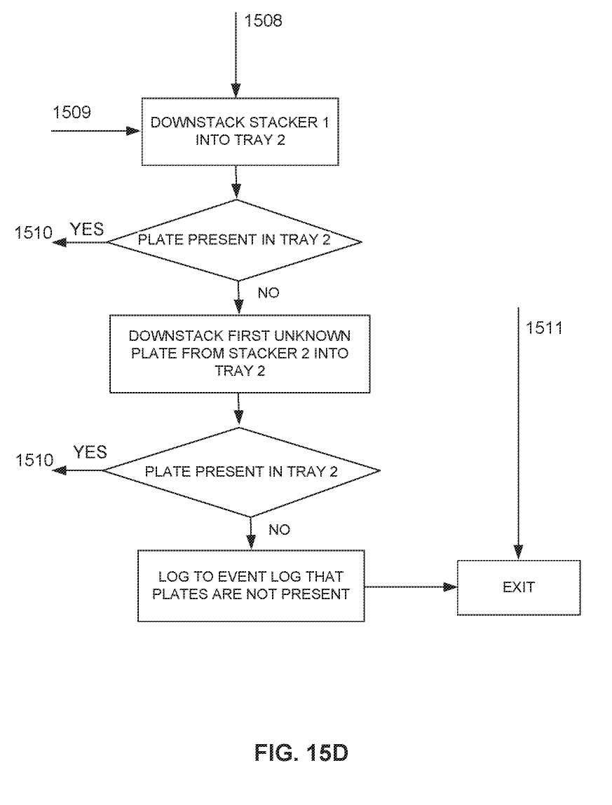

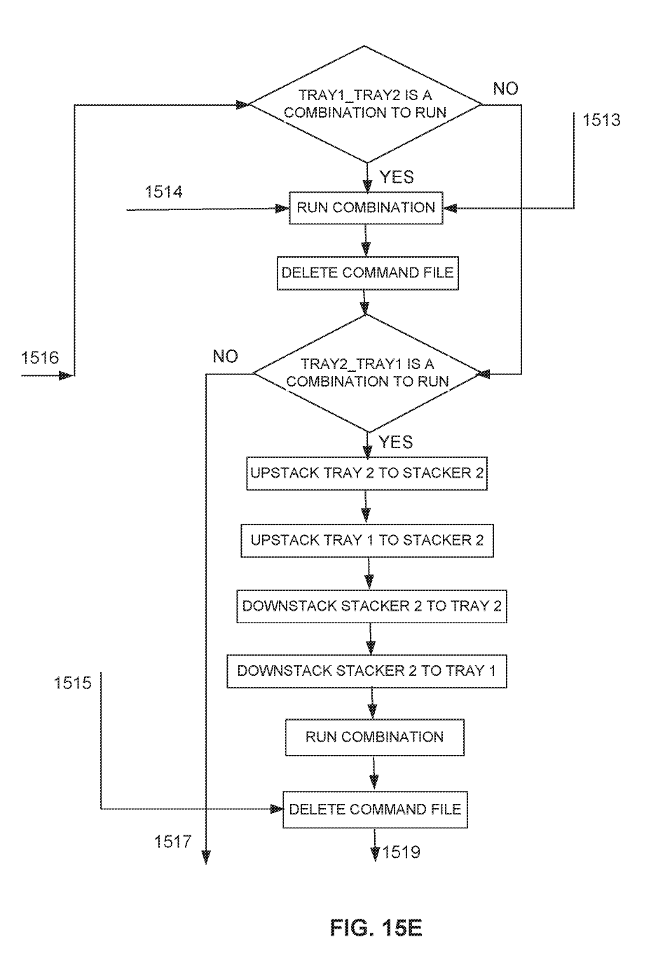

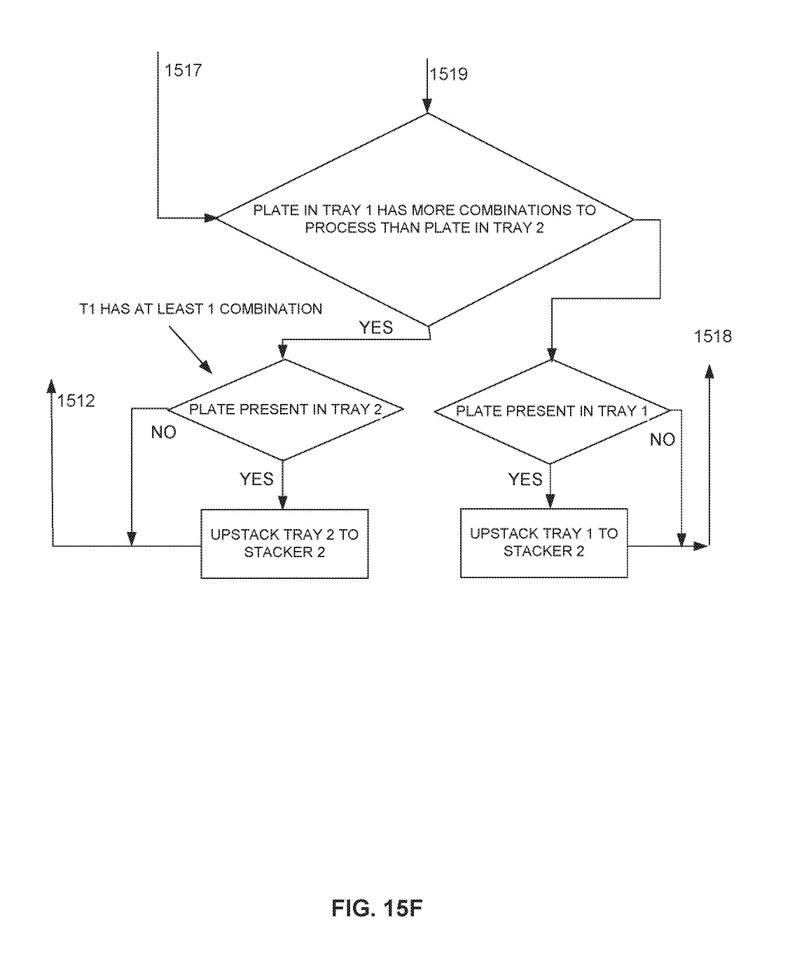

FIGS. 15A-F is a flowchart showing a first portion of a method for plate stacking, in accordance with various embodiments.

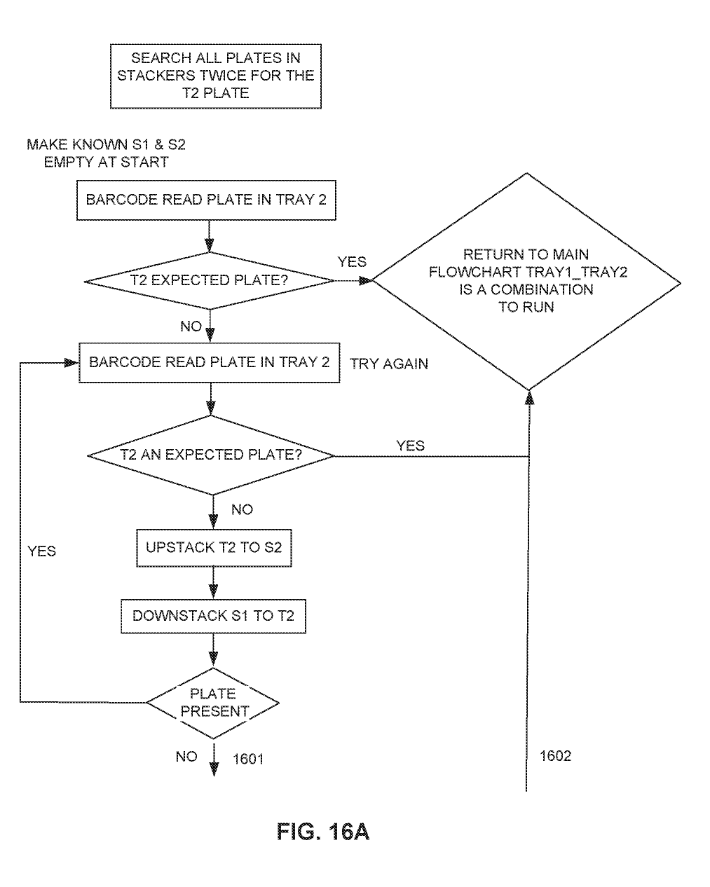

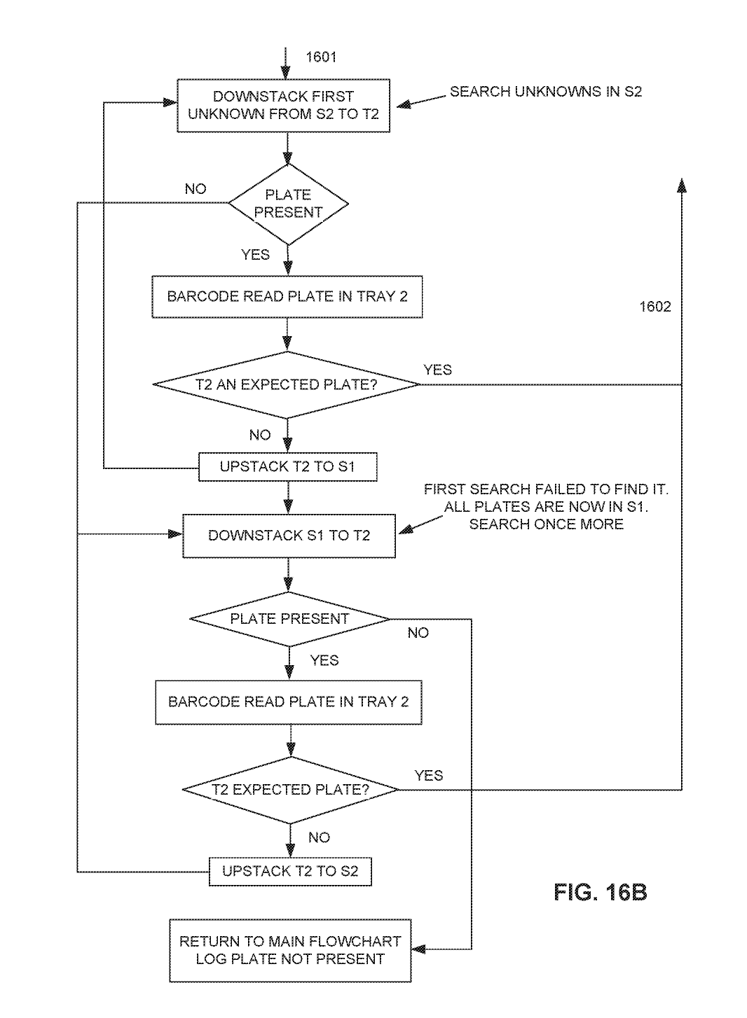

FIGS. 16A-B is a flowchart showing a second portion of a method for plate stacking, in accordance with various embodiments.

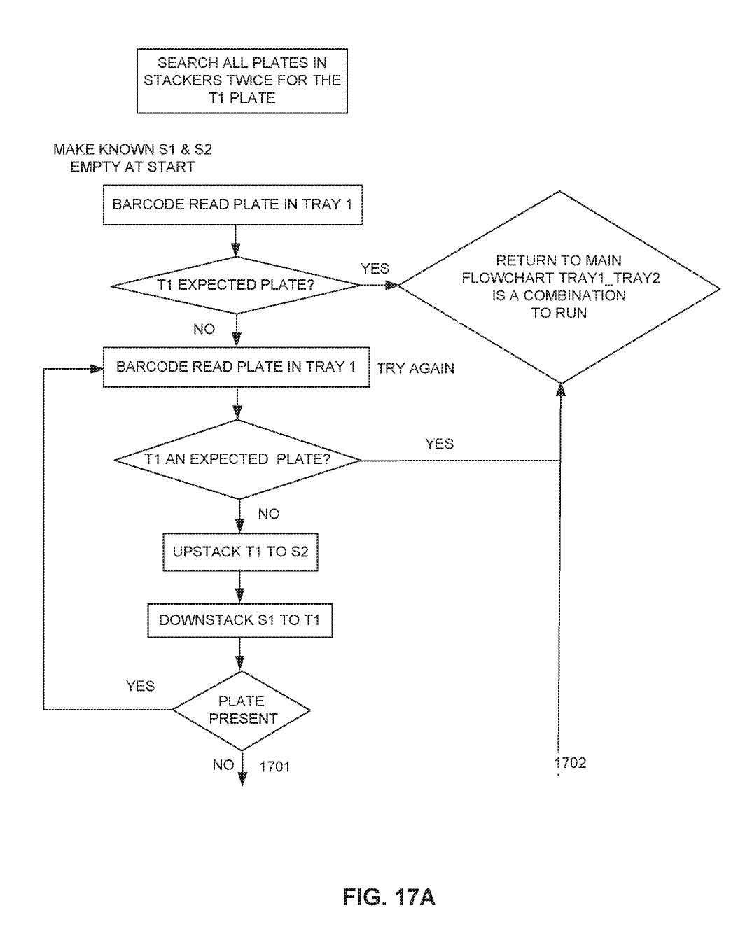

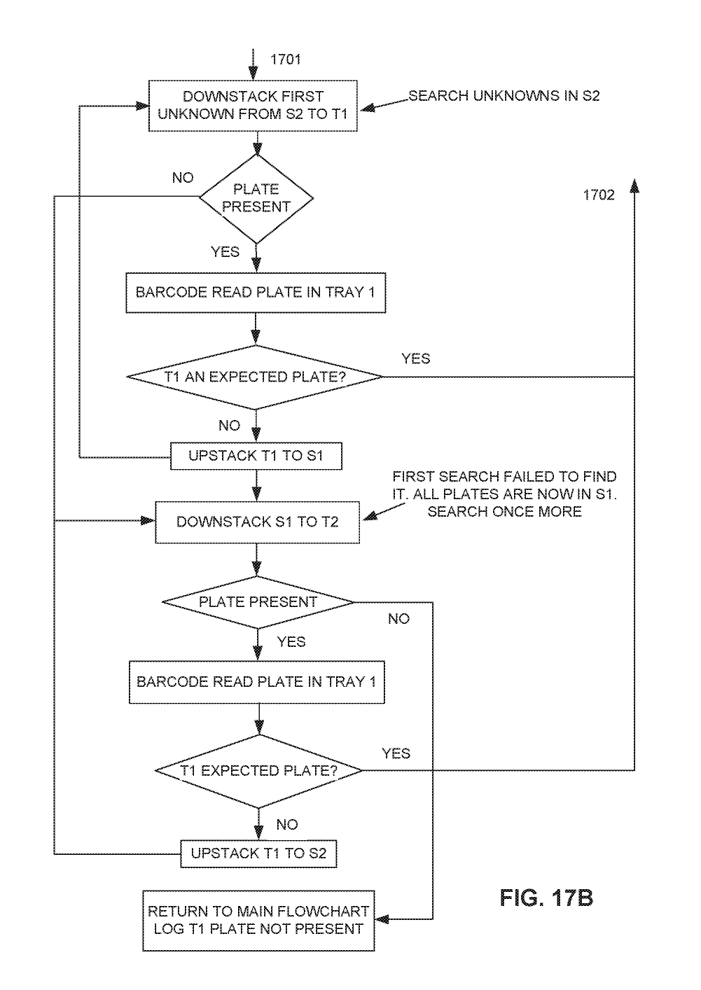

FIGS. 17A-B is a flowchart showing a third portion of a method for plate stacking, in accordance with various embodiments.

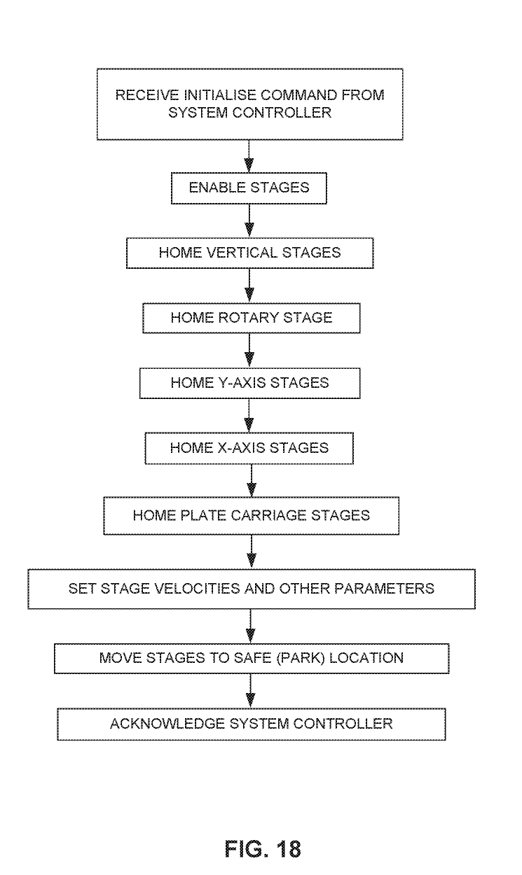

FIG. 18 is a flowchart showing a method for liquid handling initialization, in accordance with various embodiments.

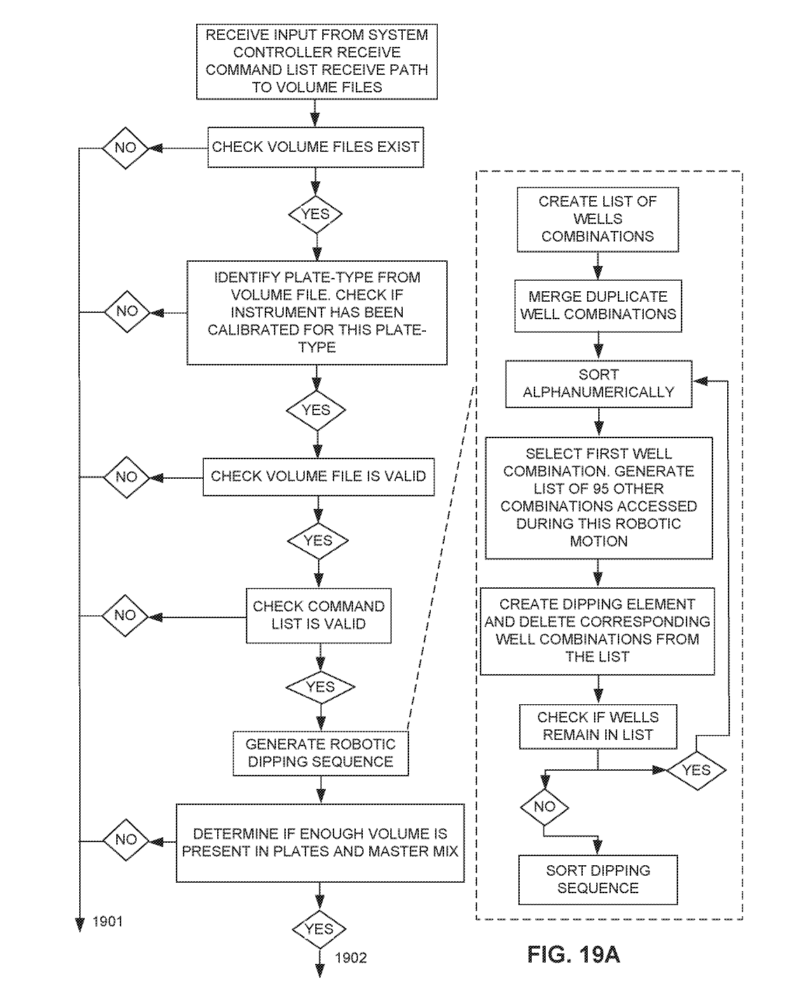

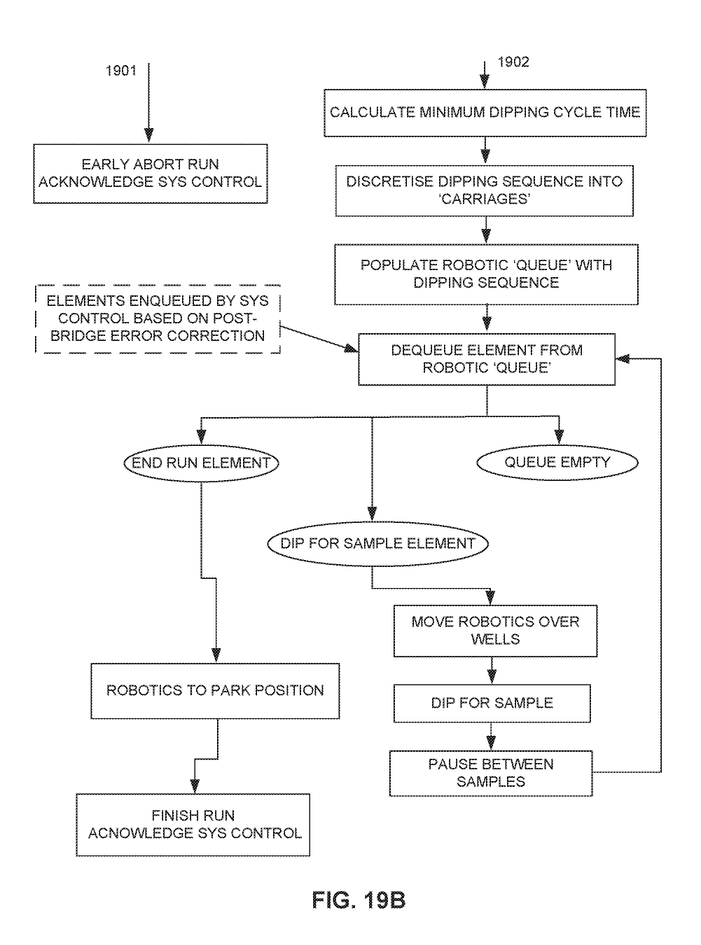

FIGS. 19A-B is a flowchart showing a method for liquid handling, in accordance with various embodiments.

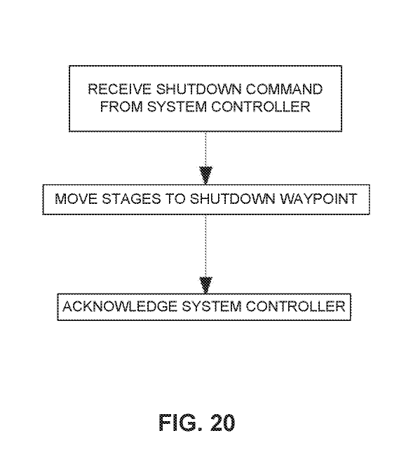

FIG. 20 is a flowchart showing a method for liquid handling shutdown, in accordance with various embodiments.

### Droplet Carriages

The droplet stream leaving the liquid bridges is divided into packets (based upon the time-stamp at which the robotics takes a sample). For convenience these packets are called carriages. The use of carriages—where the spacing between carriages is at least twice that between droplets—permits easier identification of individual droplets and indeed easy identification of errors in the droplet stream. For example droplet 2 of carriage 2 (with 5 droplets per carriage) may be identified more easily than droplet 12 of a continuous stream. Similarly errors can be easily identified. If only 4 droplets are present in a carriage of 5 then it is clear an error has occurred (droplet merging); if 6 are present then a droplet has not mixed or has mixed and then split into two.

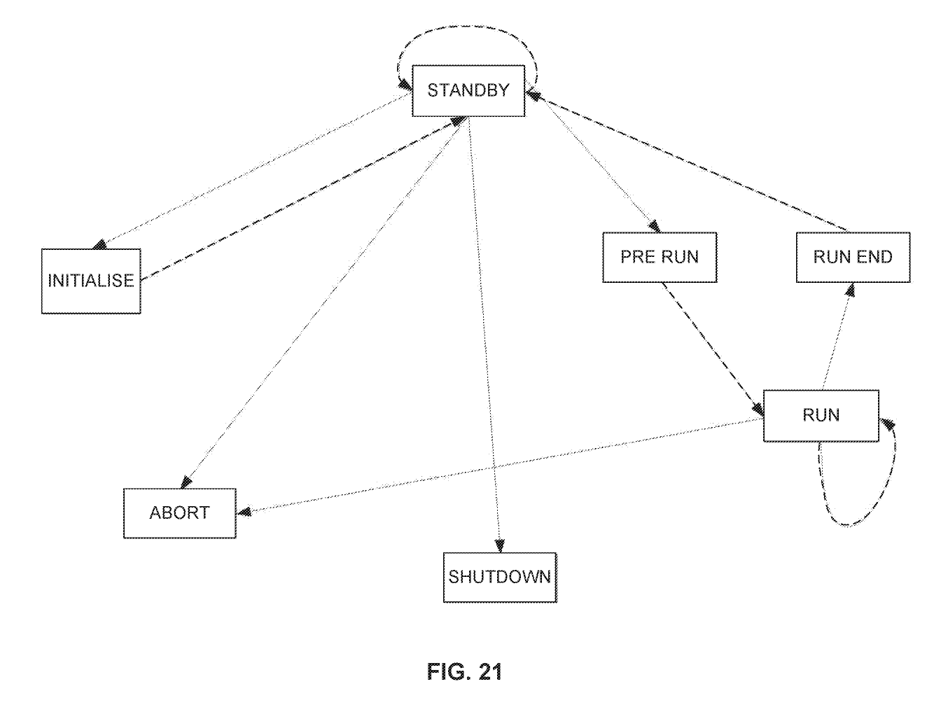

FIG. 21 is a state diagram showing the relationships among post-bridge methods, in accordance with various embodiments.

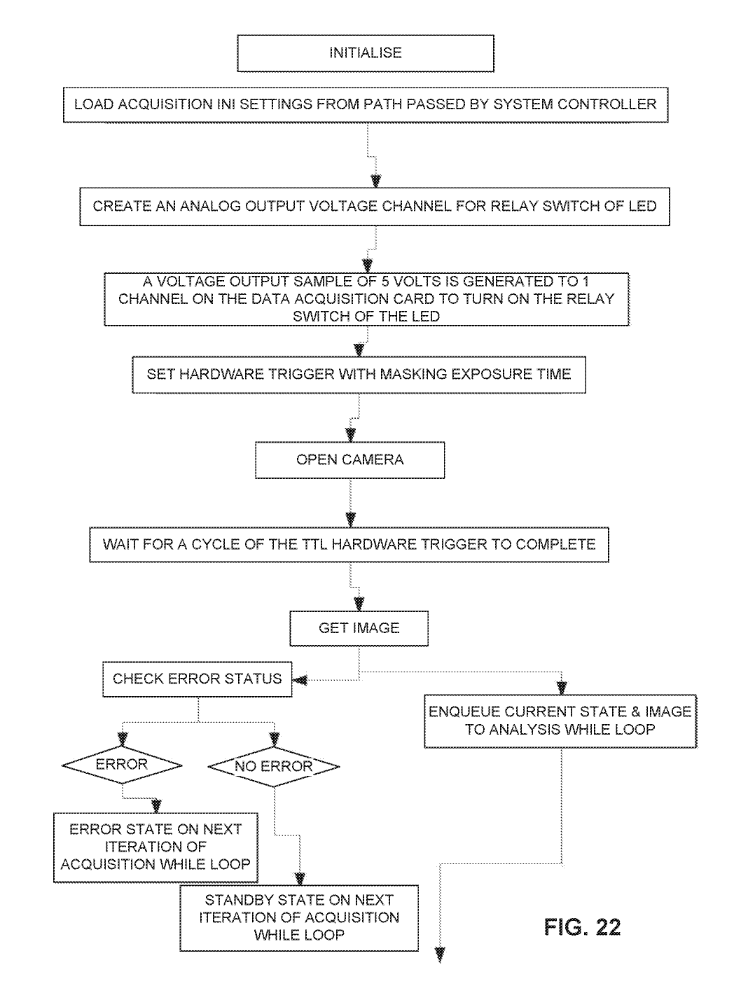

FIG. 22 is a flowchart showing a first portion of a post-bridge initialization method, in accordance with various embodiments.

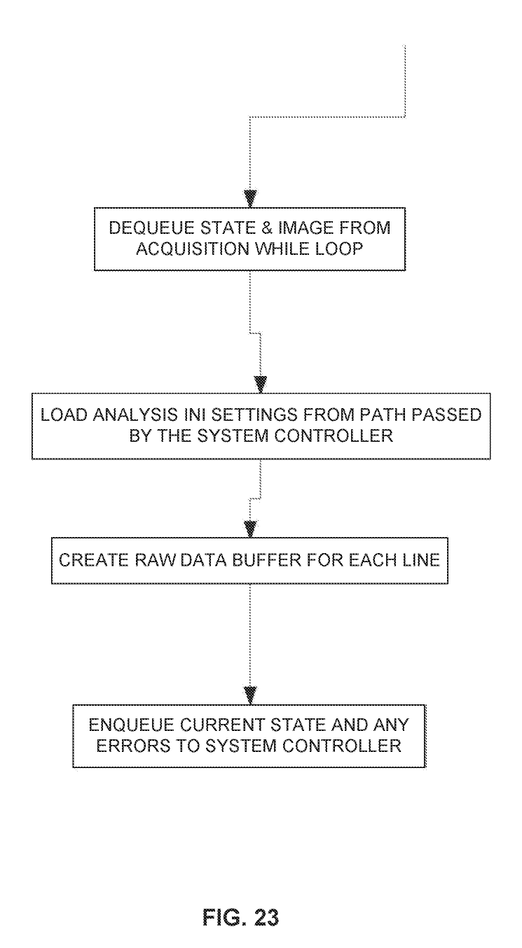

FIG. 23 is a flowchart showing a second portion of a post-bridge initialization method, in accordance with various embodiments.

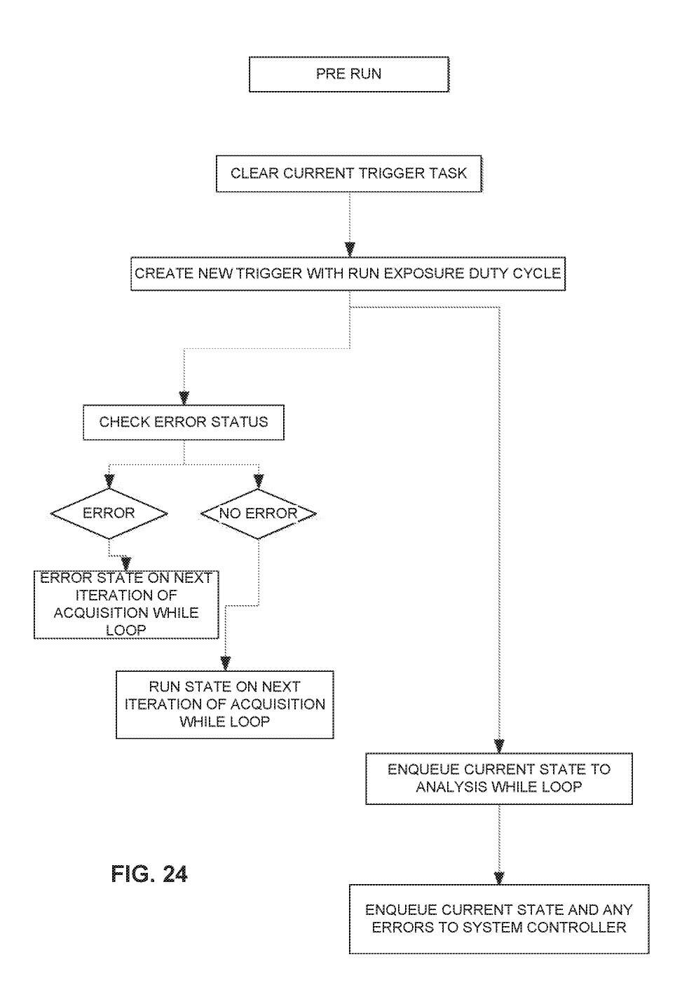

FIG. 24 is a flowchart showing a post-bridge pre run method, in accordance with various embodiments.

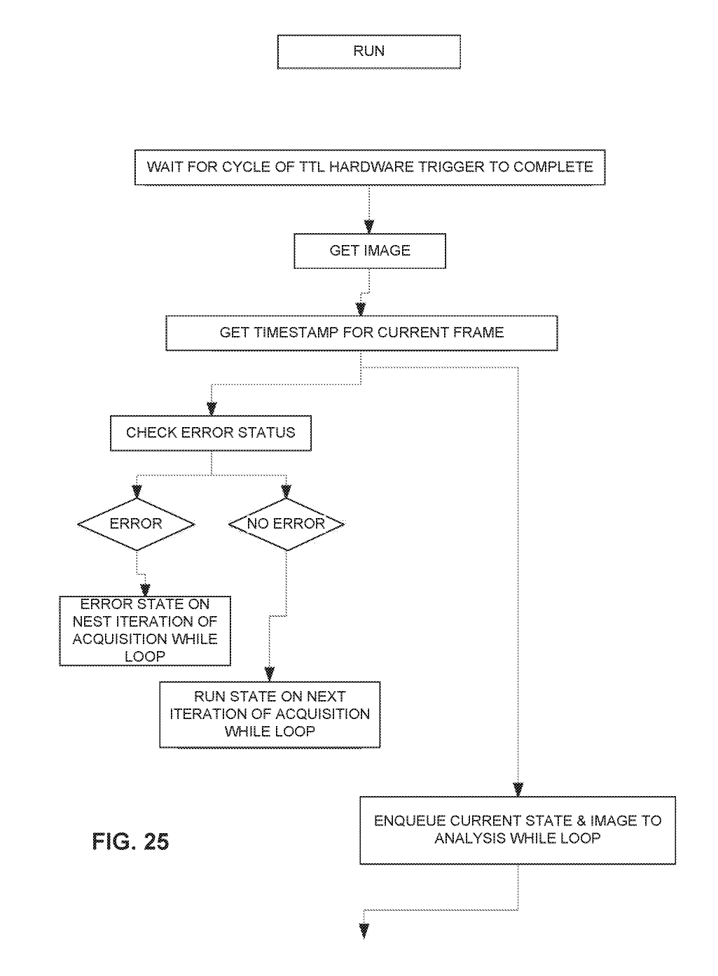

FIG. 25 is a flowchart showing a first portion of a post-bridge run method, in accordance with various embodiments.

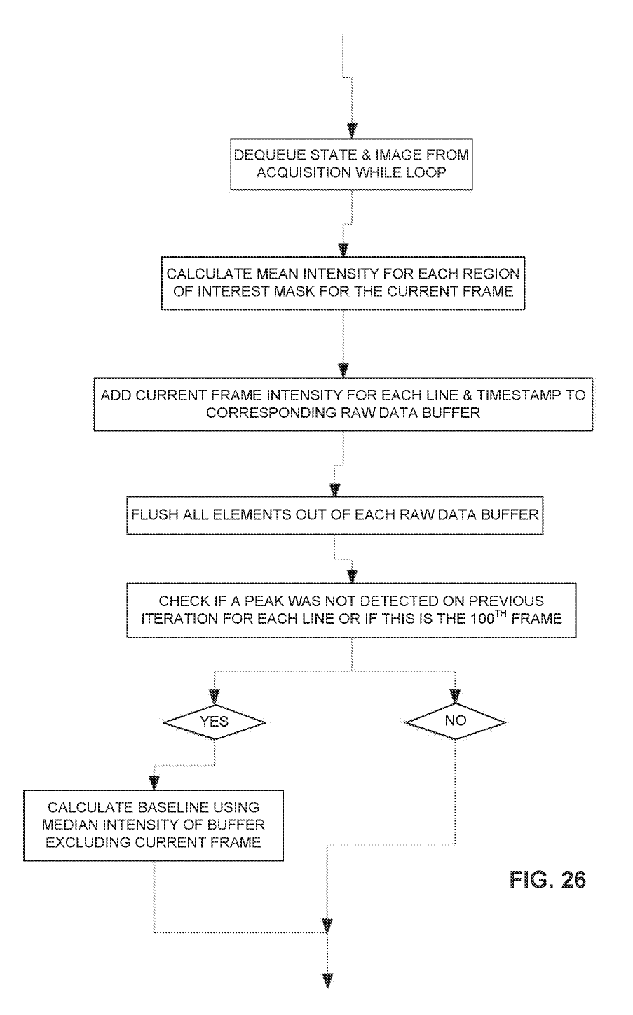

FIG. 26 is a flowchart showing a second portion of a post-bridge run method, in accordance with various embodiments.

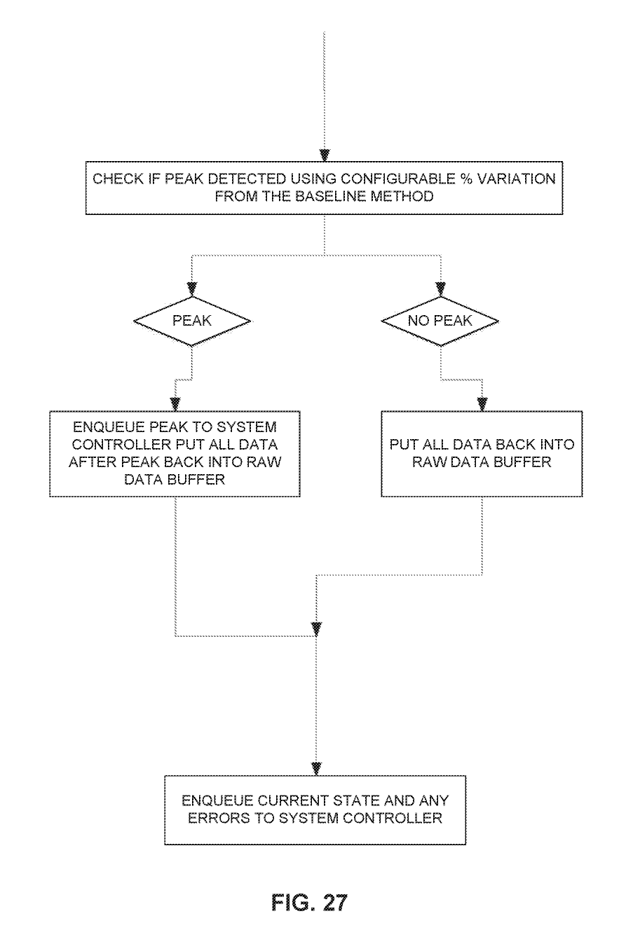

FIG. 27 is a flowchart showing a third portion of a post-bridge run method, in accordance with various embodiments.

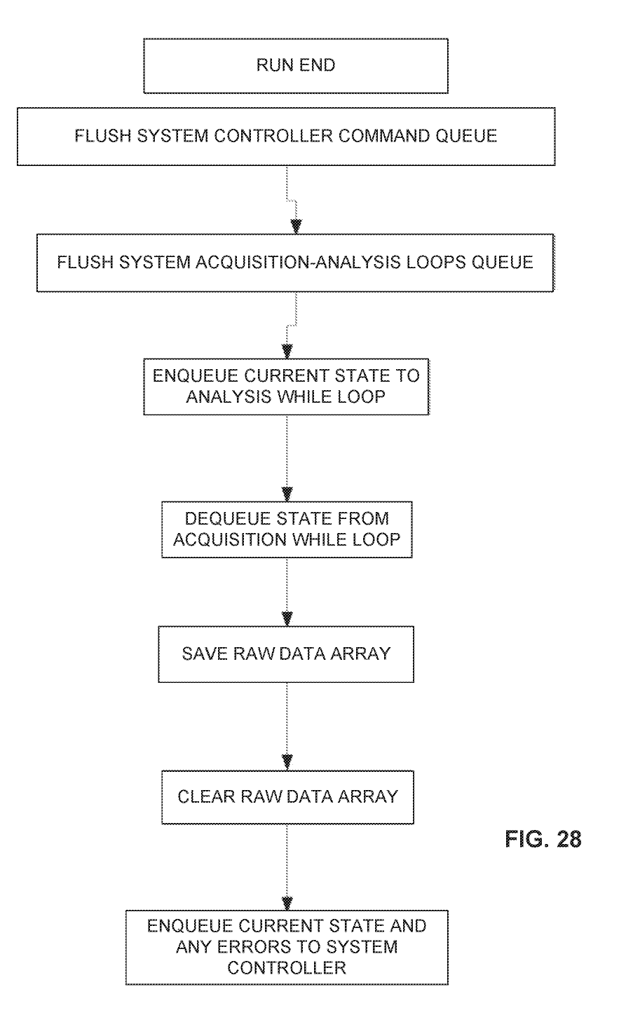

FIG. 28 is a flowchart showing a post-bridge run end method, in accordance with various embodiments.

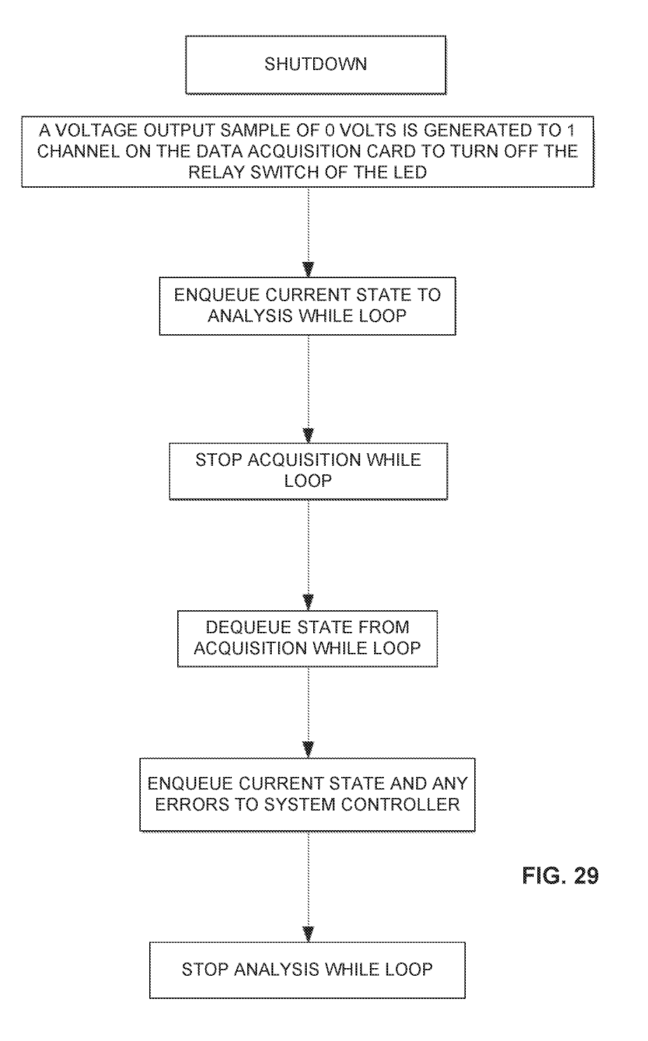

FIG. 29 is a flowchart showing a post-bridge shutdown method, in accordance with various embodiments.

### Post-Bridge Detection

The post-bridge detection system consists of an array of blue light emitting diodes (LEDs) illuminating the output line from the bridges (between the liquid bridges and the thermocycler). Three cameras (Basler) are used to monitor three fluorescent wavelengths excited by the blue LEDs. These components are FAM/VIC in the primer-probes, ROX in the Master-Mix and a third dye (i.e. ALEXA) added to the samples as a reference. If the detection system picks up all three wavelengths from a droplet, then this is considered a mixed and valid droplet. However in some cases the bridges will not mix a droplet correctly. This is found by determining that one or more of the components are missing from the main droplet. In the event an error occurs with a single droplet (or carriage) then this droplet (or the entire carriage) will be re-sampled.

### Thermocycler

The thermocycler includes four 24-line thermocyclers. Each block is preceded by a pre-heat block. Each block is maintained at its set-point using proportional integrated derivative (PID) control.

### Endpoint Detection and Analysis

Endpoint detection consists of a free-space spectrograph system. The acquisition hardware is a Hamamatsu Orca camera. The 96 thermocycler lines are illuminated by a 488 nm laser-line. This laser-line is imaged by the spectrograph/camera and resolved into its constituent wavelengths. Appropriate wavelengths are measured according to the contents of the droplets. Droplets are identified based upon the time-stamp generated by the post-bridge detection module and raw fluorescent data is generated for droplet. Spectral compensation is then applied to compensate for dye bleed through.

### File Inputs/Outputs

The PCR instrument is driven using two different ASCII .csv files. The command file is titled in the format BARCODETRAY1_BARCODETRAY2_cmds.csv while the volume file is titled BARCODETRAY1_vols.csv. The command file contains a list of well combinations which are sampled by the instrument. The volume file contains information pertaining to the contents (volume and components) of each well on the plate. On receiving a RUN command the instrument reads the barcodes of each plate present. It searches for matching command and volume files and, if present, processes this project. Results are outputted in the form BARCODETRAY1_BARCODETRAY2_rslts.csv.

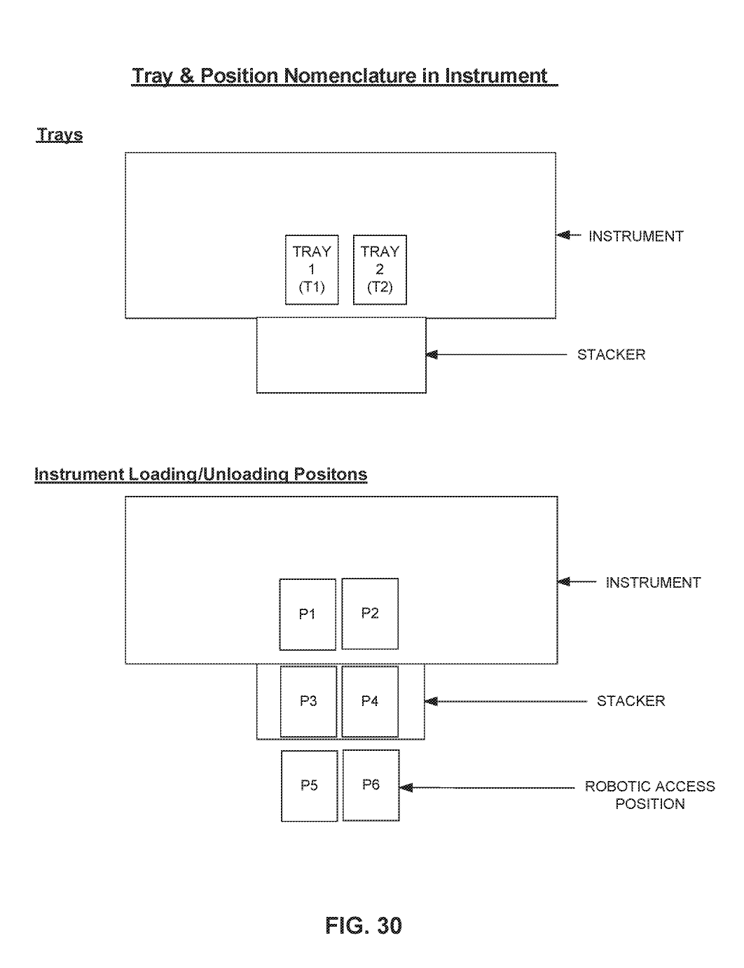

FIG. 30 is a schematic diagram showing tray and position waypoints, in accordance with various embodiments. In FIG. 30 liquid waypoints P1 through to P6 are shown. Both trays T1 and T2 can access all six waypoints. P1 and P6 are not used, for example. P2 is used for barcode reading. P3 is used for upstack/downstack into Hotel 1 on the plate-changer. P4 is used similarly for Hotel 2. P5 is used by robots to load and unload plates.

### Graphical User Interface (GUI)

A matrix of sample and reagent wells is provided to a continuous flow PCR instrument by a laboratory information management system, for example. In various embodiments, a matrix of sample and reagent wells is entered through a GUI. The GUI and the instrument interact to control the plate stacker and also to transfer files. To transfer files a file transfer protocol (FTP) setup is used. There is an FTP server that stores files and waits for clients to connect to it. The GUI acts as a client to connect to the FTP server and transfer files. The instrument can also connect to the same FTP server and transfer files.

To control the plate stacker a custom control protocol (TCP) interface is used. The instrument acts as a server and waits for the GUI to connect to it. After a connection is established predefined TCP commands are sent and received to control the instrument.

FIG. 31 is a schematic diagram showing how files are transferred between a graphical user interface (GUI) and an instrument, in accordance with various embodiments. Command files and volume files can be created and modified using the GUI. These files can then be transferred to the instrument. The files are transferred using an FTP server.

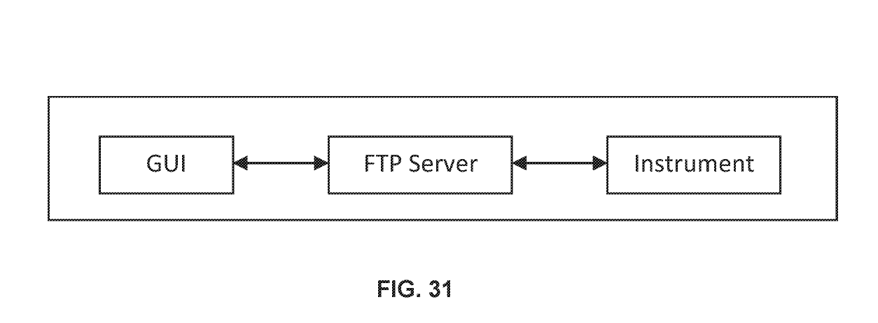

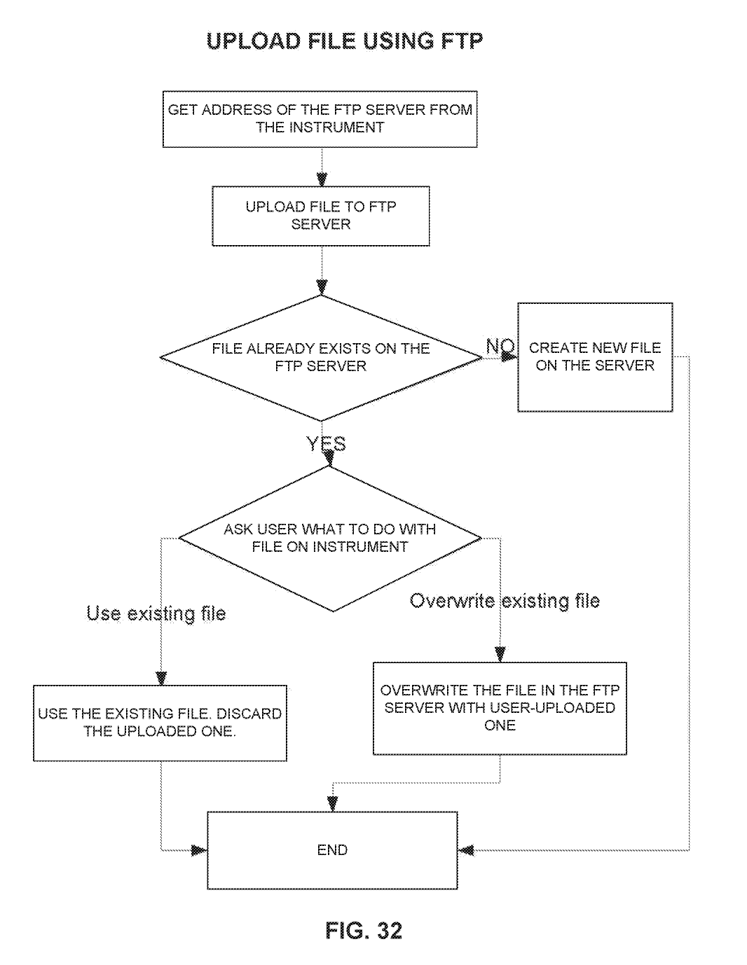

FIG. 32 is a flowchart showing a method for uploading a file using a file transfer protocol (FTP) server, in accordance with various embodiments. To upload a file, the GUI sends a TCP command to the instrument asking it for the address of the FTP server. Once the instrument has responded with this information, the GUI connects to the instrument and uploads a file. If the file already exists on the FTP server the user is asked if they want to keep it or overwrite it.

To download a file, the GUI sends a TCP command to the instrument asking it for the address of the FTP server. Once the instrument has responded with this information, the GUI connects to the instrument and presents a list of files available for downloading. The user selects a file, and the GUI then downloads it to a predefined location on the local computer.

The plate stacker allows the user of the instrument to load multiple plates at once and run them without having to explicitly load and run each plate combination individually. The stacker is divided into two compartments. Each compartment is loaded with plates. At run time the user tells the GUI which combinations to run. The GUI does not know which plates are in the stacker. Through a series of TCP commands instructing the instrument to transfer plates between the stacker and the instrument proper and to barcode the plates, the GUI can instruct the instrument to run all the selected combinations.

In various embodiments, a command file is a file that defines well combinations between plates, for example. An FTP server is a repository for files. The FTP server can communicate with the GUI and the instrument. A GUI sends commands to the instrument and creates files that can be stored on an FTP server. The instrument runs plates, receives commands from GUI, and interacts with an FTP server. A plate stacker is a component of the instrument that holds plates that are to be run on the instrument. TCP is a protocol that allows sending of information over a network. It is used between the GUI and the instrument. A volume file is a file that defines a plate. It contains the plate barcode, plate type, and volumes of wells.

### Endpoint Detection System

In order to maintain the high throughput of a continuous flow PCR system, the PCR system needs to be able to detect fluorescence in two or more micro-channels at the same time. Measuring fluorescence across two or more micro-channels imposes a number of limitations on an endpoint detection system.

For example, as the number of number of micro-channels is increased, the field of view of the detector also needs to increase. These micro-channels can be closely bundled or aligned together in an array of transparent micro-channels or tubes. However, a wall of some thickness has to be maintained between tubes to prevent crosstalk between adjacent micro-channels. As a result, the field of view of the detector is a function of the tube diameter and tube array wall thickness. In order to maintain a high fluorescence collection efficiency from the tubes on the edges of the tube array, an increased beam length can be used. Increasing the beam length from the tube array to the detector increases the overall physical size of the endpoint detection system, however.

Also, a laser is a typical illumination source for fluorescence measurements. The power distribution of a laser beam is highly non-uniform. This power distribution generally follows a Gaussian distribution and drops exponentially off-axis. However, an amplification system of a continuous flow PCR system needs an illumination source with a uniform power distribution to illuminate the entire width of the tube array.

Finally, because the flow of samples is continuous in the tube array, the PCR system has to be able to detect spectral information from two or more micro-channels in a single time step. However, in order to assign that spectral information to the correct sample, the particular tube emitting that spectral information needs to be located in the tube array. As result, the endpoint detection system needs to provide spatial information in addition to spectral information.

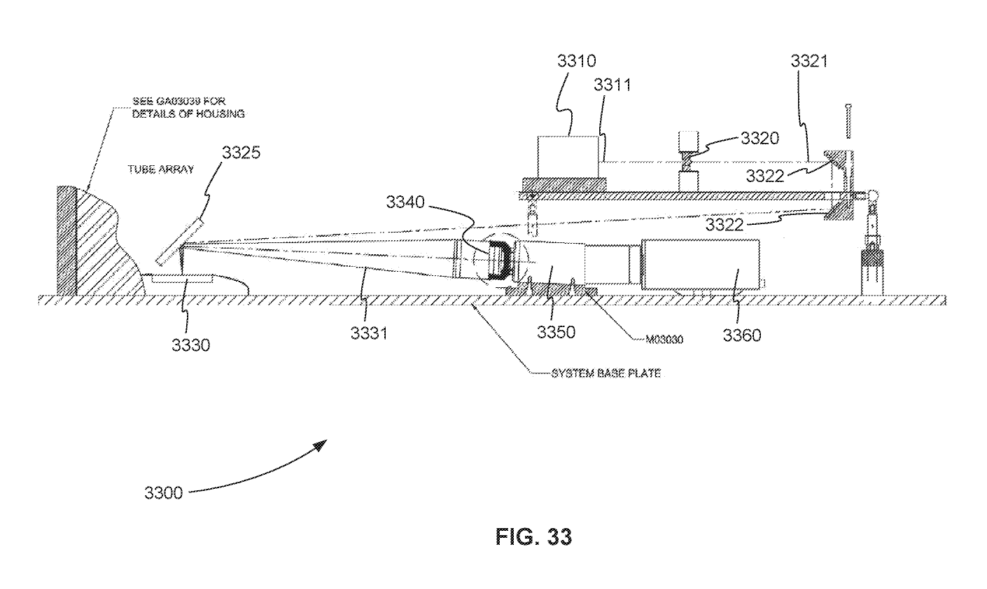

FIG. 33 is a schematic diagram of a side view of a system 3300 for detecting spectral and spatial information in a continuous flow PCR system, in accordance with various embodiments. System 3300 includes laser 3310, line generator 3320, tube array 3330, imaging lens 3340, spectrograph 3350, and imager 3360. Laser 3310 emits incident beam of electromagnetic radiation 3311.

Line generator 3320 receives incident beam 3311 from laser 3310. Line generator 3320 transforms incident beam 3311 into incident line of electromagnetic radiation 3321. On other words, line generator 3320 converts the power distribution of incident beam 3311 from a non-uniform distribution to a uniform distribution. Line generator 3320 is a Powell lens, for example. In various embodiments, line generator 3320 is a diffractive line generator.

Tube array 3330 receives incident line 3321 from line generator 3320. Tube array 3330 includes one or more transparent tubes in fluid communication with one or more micro-channels of a PCR system. In various embodiments, one or more optical elements 3322 are placed between line generator 3320 and tube array 3320 to steer incident line 3321 from line generator 3320 to tube array 3330. As shown in FIG. 33, one or more optical elements 3322 allow system 3300 to be package in an overall smaller volume, for example. In various embodiments, mirror 3325 is also placed between line generator 3320 and tube array 3330 to steer incident line 3321 from line generator 3320 to tube array 3330. Mirror 3325 allows tube array 3330 to be positioned horizontally in system 3300, for example.

Imaging lens 3340 receives reflected electromagnetic radiation 3331 from tube array 3330 and focuses reflected electromagnetic radiation 3331. In various embodiments, one or more optical elements (not shown) are placed between tube array 3330 and imaging lens 3340 to steer reflected electromagnetic radiation 3331 from tube array 3330 to imaging lens 3340. In various embodiments, mirror 3325 is placed between tube array 3330 and imaging lens 3340 to steer reflected electromagnetic radiation 3331 from tube array 3330 to imaging lens 3340. Imaging lens 3340 is a wide-iris lens with a variable aperture, for example. In various embodiments, imaging lens 3340 includes one or more optical filters (not shown). The one or more optical filters remove reflection of incident line 3321 from reflected electromagnetic radiation 3331, for example.

Spectrograph 3350 receives the focused reflected electromagnetic radiation (not shown) from the imaging lens 3340. Spectrograph 3350 detects a spectral intensity from the focused reflected electromagnetic radiation. Spectrograph 3350 can detect spectral wavelengths between 400 and 800 nanometers, for example.

Imager 3360 receives the focused reflected electromagnetic radiation from imaging lens 3340. Imager 3360 detects a location of the spectral intensity. Imager 3360 is a CCD camera, for example.

In various embodiments, system 3300 also includes a processor (not shown). The processor receives the spectral intensity from spectrograph 3350 and receives the location from imager 3360. The processor determines an intensity value for a sample moving through tube array 3330 from the spectral intensity and the location.

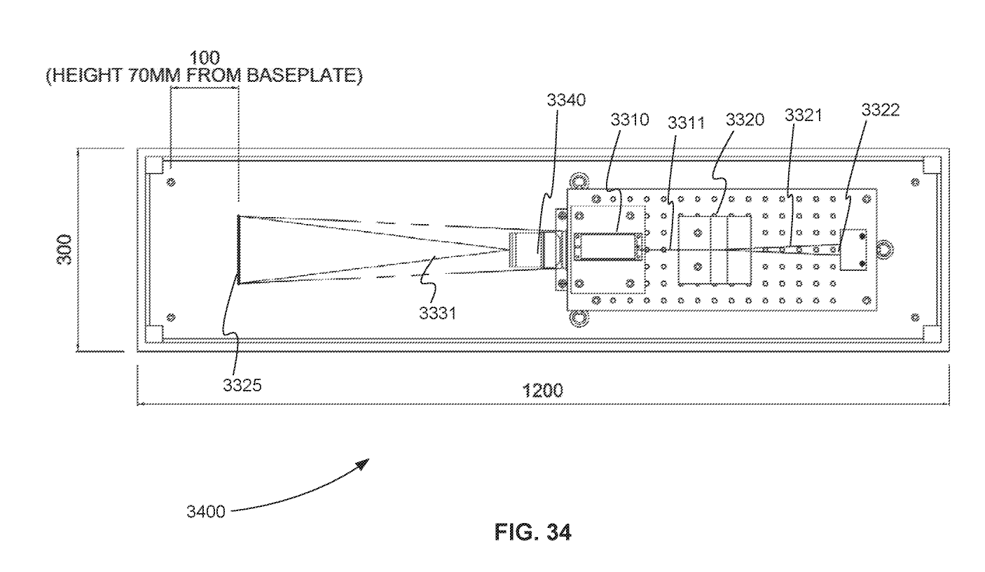

FIG. 34 is a schematic diagram of a top view of a system 3400 for detecting spectral and spatial information in a continuous flow PCR system, in accordance with various embodiments.

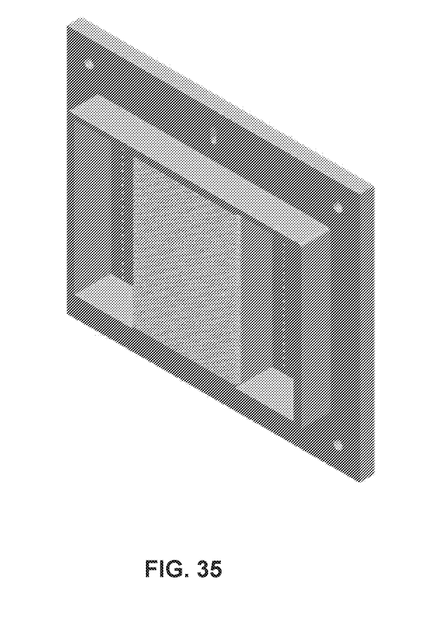

FIG. 35 is a schematic diagram of a three-dimensional view of a tube array plate, in accordance with various embodiments.

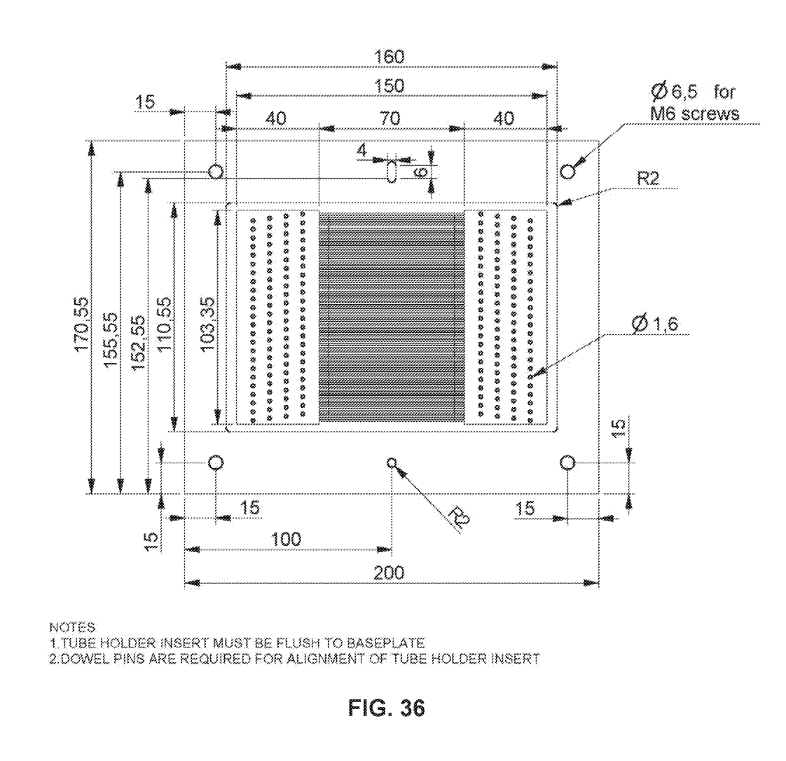

FIG. 36 is a schematic diagram of a top view of a tube array plate, in accordance with various embodiments.

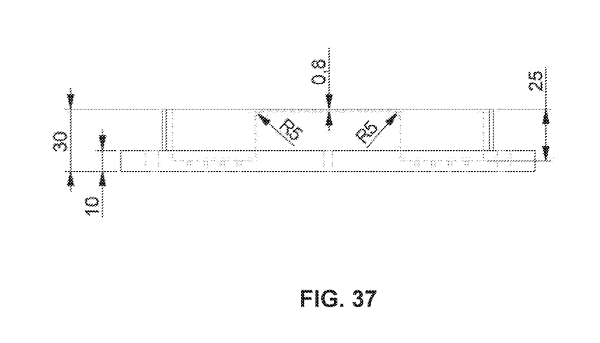

FIG. 37 is a schematic diagram of a side view of a tube array plate, in accordance with various embodiments.

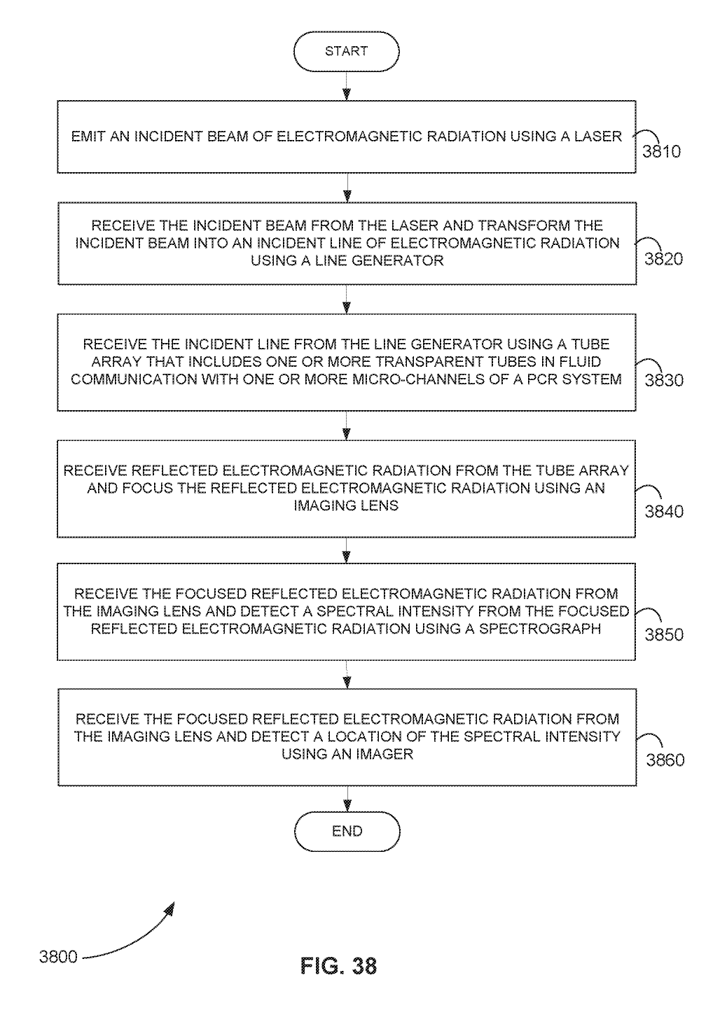

FIG. 38 is a flowchart showing a method 3800 for detecting spectral and spatial information in a continuous PCR system, in accordance with various embodiments.

In step 3810 of method 3800, an incident beam of electromagnetic radiation is emitted using a laser.

In step 3820, the incident beam is received from the laser and the incident beam is transformed into an incident line of electromagnetic radiation using a line generator.

In step 3830, the incident line is received from the line generator using a tube array that includes one or more transparent tubes in fluid communication with one or more micro-channels of a PCR system.

In step 3840, reflected electromagnetic radiation is received from the tube array and the reflected electromagnetic radiation is focused using an imaging lens.

In step 3850, the focused reflected electromagnetic radiation is received from the imaging lens and a spectral intensity is detected from the focused reflected electromagnetic radiation using a spectrograph.

In step 3860, the focused reflected electromagnetic radiation is received from the imaging lens and a location of the spectral intensity is detected using an imager.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A system for high throughput polymerase chain reaction (PCR) amplification and analysis, comprising:
a PCR system; and
a processor in communication with the PCR system that
instructs a liquid handling system of the PCR system to obtain a plurality of samples and a plurality of reagents for a PCR experiment,
instructs a fluid pumping system of the PCR system to maintain a continuous flow of a transport fluid through a plurality of micro-channels in order to receive the plurality of samples and the plurality of reagents from the liquid handling system as droplets in the plurality of micro-channels and mix the plurality of samples and the plurality of reagents using the geometry of the plurality of micro-channels producing a plurality of mixed sample droplets in the plurality of micro-channels,
receives from a post-bridge detection system of the PCR system one or more post-bridge detection values for each mixed sample droplet of the plurality of mixed sample droplets to determine if the each mixed sample droplet is mixed correctly,
instructs a thermocycler of the PCR system to maintain one or more temperatures for cycling the temperature of the plurality of mixed sample droplets in the plurality of micro-channels, and
receives from an endpoint detection system of the PCR system one or more endpoint detection values for each mixed sample droplet of the plurality of mixed sample droplets to analyze the PCR experiment.

2. The system of claim 1, wherein the processor instructs the liquid handling system to obtain a plurality of samples and a plurality of reagents for a PCR experiment by instructing the liquid handling system to pipette samples from a first sample support device, pipette assay reagents from a second sample support device, and pipette a master mix reagent from a vessel.

3. The system of claim 1, wherein one or more post-bridge detection values comprise a time stamp of the each mixed sample droplet.

4. The system of claim 1, wherein one or more post-bridge detection values comprise the intensity of electromagnetic radiation absorbed or reflected by the each mixed sample droplet.

5. The system of claim 1, wherein one or more post-bridge detection values comprise a first intensity of electromagnetic radiation emitted by a first dye of a sample of the each mixed sample droplet, a second intensity of electromagnetic radiation emitted by a second dye of an assay reagent of the each mixed sample droplet, and a third intensity of electromagnetic radiation emitted by a third dye of a master mix reagent of the each mixed sample droplet.

6. The system of claim 1, wherein the processor further instructs the liquid handling system to re-sample a sample and an assay reagent of the each mixed sample droplet, if the processor determines from the one or more post-bridge detection values that the each mixed sample droplet is mixed incorrectly.

7. The system of claim 1, wherein one or more endpoint detection values comprise a location of a micro-channel of the plurality of micro-channels and a spectral intensity detected from the micro-channel.

8. A method for high throughput polymerase chain reaction (PCR) amplification and analysis, comprising:
instructing a liquid handling system of a PCR system to obtain a plurality of samples and a plurality of reagents for a PCR experiment using a processor;
instructing a fluid pumping system of the PCR system to maintain a continuous flow of a transport fluid through a plurality of micro-channels in order to receive the plurality of samples and the plurality of reagents from the liquid handling system as droplets in the plurality of micro-channels and in order to mix the plurality of samples and the plurality of reagents using the geometry of the plurality of micro-channels producing a plurality of mixed sample droplets in the plurality of micro-channels using the processor;
receiving from a post-bridge detection system of the PCR system one or more post-bridge detection values for each mixed sample droplet of the plurality of mixed sample droplets to determine if the each mixed sample droplet is mixed correctly using the processor;
instructing a thermocycler of the PCR system to maintain one or more temperatures for cycling the temperature of the plurality of mixed sample droplets in the plurality of micro-channels using the processor; and
receiving from an endpoint detection system of the PCR system one or more endpoint detection values for each mixed sample droplet of the plurality of mixed sample droplets to analyze the PCR experiment using the processor.

9. The method of claim 8, wherein instructing the liquid handling system to obtain a plurality of samples and a plurality of reagents for a PCR experiment comprises instructing the liquid handling system to pipette samples from a first sample support device, pipette assay reagents from a second sample support device, and pipette a master mix reagent from a vessel.

10. The method of claim 8, wherein one or more post-bridge detection values comprise a time stamp of the each mixed sample droplet.

11. The method of claim 8, wherein one or more post-bridge detection values comprise the intensity of electromagnetic radiation absorbed or reflected by the each mixed sample droplet.

12. The method of claim 8, wherein one or more post-bridge detection values comprise a first intensity of electromagnetic radiation emitted by a first dye of a sample of the each mixed sample droplet, a second intensity of electromagnetic radiation emitted by a second dye of an assay reagent of the each mixed sample droplet, and a third intensity of electromagnetic radiation emitted by a third dye of a master mix reagent of the each mixed sample droplet.

13. The method of claim 8, further comprising instructing the liquid handling system to re-sample a sample and an assay reagent of the each mixed sample droplet using the processor, if it is determined from the one or more post-bridge detection values that the each mixed sample droplet is mixed incorrectly.

14. The method of claim 8, wherein one or more endpoint detection values comprise a location of a micro-channel of the plurality of micro-channels and a spectral intensity detected from the micro-channel.

15. A computer program product, comprising a non-transitory and tangible computer-readable storage medium whose contents include a program with instructions being executed on a processor so as to perform a method for high throughput polymerase chain reaction (PCR) amplification and analysis, the method comprising:
providing a system, wherein the system comprises one or more distinct software modules, and wherein the distinct software modules comprise a liquid handling module, a fluid pumping module, a post-bridge detection module, a thermocycler module, and an endpoint detection module;
instructing a liquid handling system of a PCR system to obtain a plurality of samples and a plurality of reagents for a PCR experiment using the liquid handling module;
instructing a fluid pumping system of the PCR system to maintain a continuous flow of a transport fluid through a plurality of micro-channels in order to receive the plurality of samples and the plurality of reagents from the liquid handling system as droplets in the plurality of micro-channels and in order to mix the plurality of samples and the plurality of reagents using the geometry of the plurality of micro-channels producing a plurality of mixed sample droplets in the plurality of micro-channels using the fluid pumping module;
receiving from a post-bridge detection system of the PCR system one or more post-bridge detection values for each mixed sample droplet of the plurality of mixed sample droplets to determine if the each mixed sample droplet is mixed correctly using the post-bridge detection module;
instructing a thermocycler of the PCR system to maintain one or more temperatures for cycling the temperature of the plurality of mixed sample droplets in the plurality of micro-channels using the thermocycler module; and
receiving from an endpoint detection system of the PCR system one or more endpoint detection values for each mixed sample droplet of the plurality of mixed sample droplets to analyze the PCR experiment using the endpoint detection module.

16. The computer program product of claim 15, wherein instructing the liquid handling system to obtain a plurality of samples and a plurality of reagents for a PCR experiment comprises instructing the liquid handling system to pipette samples from a first sample support device, pipette assay reagents from a second sample support device, and pipette a master mix reagent from a vessel.

17. The computer program product of claim 15, wherein one or more post-bridge detection values comprise a time stamp of the each mixed sample droplet.

18. The computer program product of claim 15, wherein one or more post-bridge detection values comprise the intensity of electromagnetic radiation absorbed or reflected by the each mixed sample droplet.

19. The computer program product of claim 15, wherein one or more post-bridge detection values comprise a first intensity of electromagnetic radiation emitted by a first dye of a sample of the each mixed sample droplet, a second intensity of electromagnetic radiation emitted by a second dye of an assay reagent of the each mixed sample droplet, and a third intensity of electromagnetic radiation emitted by a third dye of a master mix reagent of the each mixed sample droplet.

20. The computer program product of claim 15, further comprising instructing the liquid handling system to re-sample a sample and an assay reagent of the each mixed sample droplet using the liquid handling module, if it is determined from the one or more post-bridge detection values that the each mixed sample droplet is mixed incorrectly.
